(19) United States
(12) Patent Application Publication
(10) Pub. No.: US 2006/0270684 A1
(43) Pub. Date: Nov. 30, 2006

(54) Crystalline forms of ziprasidone mesylate

(76) Inventors: Judith Aronhime, Rehovot (IL); Marioara Mendelovici, Rechovot (IL); Sigalit Levi, Modi'in (IL); Alex Mainfeld, Herzilia (IL)

Correspondence Address: KENYON & KENYON LLP, ONE BROADWAY, NEW YORK, NY, 10004, US

(21) Appl. No.: 11/353,421

(22) Filed: Feb. 13, 2006

Related U.S. Application Data
(60) Provisional application No. 60/661,687, filed on Mar. 14, 2005.
(60) Provisional application No. 60/689,701, filed on Jun. 9, 2005.
(60) Provisional application No. 60/705,762, filed on Aug. 4, 2005.
(60) Provisional application No. 60/762,349, filed on Jan. 25, 2006.
(60) Provisional application No. 60/762,695, filed on Jan. 26, 2006.

(51) Int. Cl. A61K 31/496; C07D 417/14

(52) U.S. Cl. 514/254.06; 544/368

(57) ABSTRACT

Provided are polymorphic forms of ziprasidone mesylate and processes for their preparation.

### RELATED APPLICATIONS

This application claims the benefit of U.S. Provisional Application Nos. 60/661,687, filed Mar. 14, 2005, 60/689,701, filed Jun. 9, 2005, 60/705,762, filed Aug. 4, 2005, 60/762,349 filed Jan. 25, 2006 and 60/762,695 filed Jan. 26, 2006, all of which are herein incorporated by reference.

### FIELD OF INVENTION

The present invention is directed to novel ziprasidone mesylate polymorphs and process for preparing ziprasidone mesylate polymorphs.

### BACKGROUND OF THE INVENTION

Ziprasidone is an antipsychotic agent and is therefore useful for treating various disorders including schizophrenia, anxiety and migraine pain. Ziprasidone has the following structure:

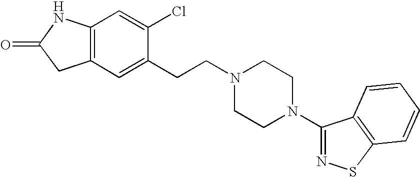

Ziprasidone has been marketed under the name GEODON as an oral capsule and as an injectable drug. GEODON capsules contain the monohydrate hydrochloride salt of ziprasidone, and come in 20, 40, 60 and 80 mg dosage forms. GEODON for injection contains a lyophilized form of ziprasidone mesylate trihydrate, and contains 20 mg base equivalent of ziprasidone.

The present invention relates to the solid state physical properties of ziprasidone mesylate. These properties may be influenced by controlling the conditions under which ziprasidone mesylate is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate. Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and may be used to distinguish some forms from others. A particular form may also give rise to distinct spectroscopic properties that may be detectable by powder X-ray crystallography, solid state ¹³C NMR spectrometry and infrared spectrometry.

The preparation of ziprasidone base is disclosed in U.S. Pat. No. 4,831,031 (example 16). Preparation of ziprasidone base is also disclosed in U.S. Pat. No. 5,312,925. U.S. Pat. No. 6,245,765 discloses dihydrate crystalline salts of ziprasidone mesylate and their use as dopamine antagonists. U.S. Pat. No. 6,110,918 discloses four known ziprasidone mesylate crystalline forms: anhydrous (lath crystal), dihydrate (lath crystal), dihydrate (needle crystal) and trihydrate (prism crystal). Each crystal form may be characterized by a distinct X-ray powder diffraction pattern and a distinct crystal shape that can be observed by photomicrograph. U.S. Pat. No. 6,110,918 also reports that the ziprasidone mesylate dihydrate lath crystals and dihydrate needle crystals are relatively long and thin in contrast to the prism crystals of ziprasidone mesylate trihydrate. In an aqueous medium at ambient temperature, ziprasidone mesylate trihydrate is reported to be the most thermodynamically stable form of the four crystalline forms of ziprasidone mesylate. U.S. Pat. No. 6,399,777 discloses the preparation of ziprasidone mesylate anhydrous lath forms by slurrying ziprasidone base in isopropyl alcohol. Publication No. US 2004/0194338 discloses a multitude of dispersions containing amorphous drugs and polymers, prepared by spray drying.

The discovery of new forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional forms of ziprasidone mesylate and/or processes for their preparation.

### SUMMARY OF THE INVENTION

One embodiment of the invention encompasses ziprasidone mesylate crystal form, characterized by X-ray powder diffraction peaks at about 11.7, 17.3, 23.5, 24.2 and 25.2 degrees two-theta, ±0.2 degrees two-theta.

Another embodiment of the invention encompasses ziprasidone mesylate crystal form, characterized by X-ray powder diffraction peaks at about 17.1, 18.8, 21.0 and 23.7 degrees two-theta, ±0.2 degrees two-theta.

Another embodiment of the invention encompasses ziprasidone mesylate crystal form, characterized by X-ray powder diffraction peaks at about 20.9, 21.3, 24.0, 24.5 and 25.8 degrees two-theta, ±0.2 degrees two-theta.

Another embodiment of the invention encompasses ziprasidone mesylate crystal form, characterized by X-ray powder diffraction peaks at about 17.1, 18.9, 22.7, 23.6 and 24.3 degrees two-theta, ±0.2 degrees two-theta.

Another embodiment of the invention encompasses ziprasidone mesylate crystal form, characterized by X-ray powder diffraction peaks at about 22.1, 25.5, 26.8, 27.1 and 27.5 degrees two-theta, ±0.2 degrees two-theta.

Another embodiment of the invention encompasses ziprasidone mesylate crystal form, characterized by X-ray powder diffraction peaks at about 15.1, 23.0, 23.5, and 23.8 degrees two-theta, ±0.2 degrees two-theta.

Another embodiment of the invention encompasses ziprasidone mesylate crystal form, characterized by X-ray powder diffraction peaks at about 17.2, 19.0, 21.0, 24.3, and 24.9 degrees two-theta, ±0.2 degrees two-theta.

Another embodiment of the invention encompasses ziprasidone mesylate crystal form, characterized by X-ray powder diffraction peaks at about 17.1, 18.7, 23.8, and 24.4 degrees two-theta, ±0.2 degrees two-theta.

Another embodiment of the invention encompasses ziprasidone mesylate crystal form, characterized by X-ray powder diffraction peaks at about 17.1, 18.7, 20.9, 23.8 and 24.3 degrees two-theta, ±0.2 degrees two-theta.

Another embodiment of the invention encompasses ziprasidone mesylate crystal form, characterized by X-ray powder diffraction peaks at about 7.8, 15.6, 17.9, 20.0 and 23.8 degrees two-theta, ±0.2 degrees two-theta.

Another embodiment of the invention encompasses ziprasidone mesylate crystal form, characterized by X-ray powder diffraction peaks at about 17.1, 18.9, 20.9, 22.0, 23.6 and 24.6 degrees two-theta, ±0.2 degrees two-theta.

Another embodiment of the invention encompasses ziprasidone mesylate crystal form, characterized by X-ray powder diffraction peaks at about 16.9, 17.7, 19.1, 21.1, 23.0 and 24.5 degrees two-theta, ±0.2 degrees two-theta.

Another embodiment of the invention encompasses ziprasidone mesylate crystal form, characterized by X-ray powder diffraction peaks at about 16.4, 16.9, 23.7, 25.1 and 26.9 degrees two-theta, ±0.2 degrees two-theta.

Another embodiment of the invention encompasses ziprasidone mesylate crystal form, characterized by X-ray powder diffraction peaks at about 16.2, 18.8, 21.3, 24.4 and 26.1 degrees two-theta, ±0.2 degrees two-theta.

Another embodiment of the invention encompasses ziprasidone mesylate crystal form, characterized by X-ray powder diffraction peaks at about 18.5, 22.0, 23.8, 24.2 and 26.1 degrees two-theta, ±0.2 degrees two-theta.

The present invention also provides processes for preparing the above crystalline forms of ziprasidone mesylate.

Another embodiment of the invention encompasses ziprasidone mesylate solvate of any one of: acetic acid, ethanol, methanol, 1,4-dioxane, ethylene glycol or THF. Preferably, the ziprasidone mesylate solvate contains water.

Another embodiment of the invention encompasses a process for crystallizing dihydrate needle crystals from a mixture of methanesulfonic acid and a slurry of ziprasidone base and THF, at about room temperature.

Another embodiment of the invention encompasses a process for preparing ziprasidone mesylate dihydrate lath crystals, comprising drying ziprasidone mesylate Form III, Form V or Form IX.

Another embodiment of the invention encompasses a process for preparing ziprasidone mesylate anhydrous lath crystals, comprising drying ziprasidone mesylate Form IX.

Another embodiment of the invention encompasses a process for preparing a mixture of ziprasidone mesylate Form XIII and anhydrous ziprasidone mesylate lath crystals by drying ziprasidone Form II.

Also provided are pharmaceutical formulations, comprising the crystalline forms of the present invention and at least one pharmaceutically acceptable excipient.

Another aspect of the present invention is a method for treating schizophrenia comprising administering such pharmaceutical composition to a human in need thereof.

### BRIEF DESCRIPTION OF THE DRAWINGS

FIG. 1. X-Ray powder diffractogram of ziprasidone mesylate Form I.

FIG. 2. X-Ray powder diffractogram of Form I and amorphous form.

FIG. 3. TGA thermogram of ziprasidone mesylate Form I.

FIG. 4. DSC thermogram of ziprasidone mesylate Form I.

FIG. 5a. A photomicrograph of ziprasidone mesylate Form I.

FIG. 5b. A photomicrograph of ziprasidone mesylate anydrous transformed from Form I.

FIG. 6. X-Ray powder diffractogram of ziprasidone mesylate Form II.

FIG. 7. TGA thermogram of ziprasidone mesylate Form II.

FIG. 8. DSC thermogram of ziprasidone mesylate Form II.

FIG. 9. A photomicrograph of ziprasidone mesylate Form II.

FIG. 10. X-Ray powder diffractogram of ziprasidone mesylate Form III.

FIG. 11. TGA thermogram of ziprasidone mesylate Form III.

FIG. 12. DSC thermogram of ziprasidone mesylate Form III.

FIG. 13. A photomicrograph of ziprasidone mesylate Form III.

FIG. 14. X-Ray powder diffractogram of ziprasidone mesylate Form IV.

FIG. 15. TGA thermogram of ziprasidone esylate Form IV.

FIG. 16. DSC thermogram of ziprasidone mesylate Form IV.

FIG. 17. A photomicrograph of ziprasidone mesylate Form IV.

FIG. 18. X-Ray powder diffractogram of ziprasidone mesylate Form V.

FIG. 19. TGA thermogram of ziprasidone mesylate Form V.

FIG. 20. DSC thermogram of ziprasidone mesylate Form V.

FIG. 21. A photomicrograph of ziprasidone mesylate Form V.

FIG. 22. X-Ray powder diffractogram of ziprasidone mesylate Form VI.

FIG. 23. TGA thermogram of ziprasidone mesylate Form VI.

FIG. 24. DSC thermogram of ziprasidone mesylate Form VI.

FIG. 25. A photomicrogaph of ziprasidone mesylate Form VI.

FIG. 26. X-Ray powder diffractogram of ziprasidone mesylate Form VII.

FIG. 27. TGA thermogram of ziprasidone mesylate Form VII.

FIG. 28. DSC thermogram of ziprasidone mesylate Form VII.

FIG. 29. A photomicrograph of ziprasidone mesylate Form VII.

FIG. 30. X-Ray powder diffractogram of ziprasidone mesylate Form VIII.

FIG. 31. TGA thermogram of ziprasidone mesylate Form VIII.

FIG. 32. DSC thermogram of ziprasidone mesylate Form VIII.

FIG. 33. DSC thermogram of ziprasidone mesylate form amorphous.

FIG. 34. A photomicrograph of ziprasidone mesylate amorphous.

FIG. 35. X-Ray powder diffractogram of ziprasidone mesylate Form IX.

FIG. 36 X-Ray powder diffractogram of ziprasidone mesylate Form X.

FIG. 37. X-Ray powder diffractogram of ziprasidone mesylate Form XIII.

FIG. 38. DSC thermogram of ziprasidone mesylate Form IX.

FIG. 39. DSC thermogram of ziprasidone mesylate Form X.

FIG. 40. DSC thermogram of ziprasidone mesylate Form XIII.

FIG. 41. A photomicrograph of ziprasidone mesylate Form IX.

FIG. 42. A photomicrograph of ziprasidone mesylate Form X.

FIG. 43. A photomicrograph of ziprasidone mesylate Form XIII.

FIG. 44. A photomicrograph of ziprasidone mesylate Form VIII.

FIG. 45. X-ray diffractogram of ziprasidone mesylate amorphous.

FIG. 46. TGA thermogram of ziprasidone mesylate amorphous.

FIG. 47. XRD diffractogram of ziprasidone mesylate Form XVI.

FIG. 48. XRD diffractogram of ziprasidone mesylate Form XVII.

FIG. 49. XRD diffractogram of ziprasidone mesylate Form XVIII.

FIG. 50. DSC thermogram of ziprasidone mesylate Form XVIII.

FIG. 51. XRD diffractogram of ziprasidone mesylate Form XIX.

FIG. 52. DSC thermogram of ziprasidone mesylate Form XIX.

### DETAILED DESCRIPTION OF THE INVENTION

As used herein, the term “slurry” refers to a heterogeneous mixture.

As used herein, the term “non-hygroscopic” refers to a compound that does not absorb more than 0.2% of water at 80% humidity, at a temperature of 25° C. for 24 hours, as described in Pharmeuropa, Vol. 4, No. 3, September 1992.

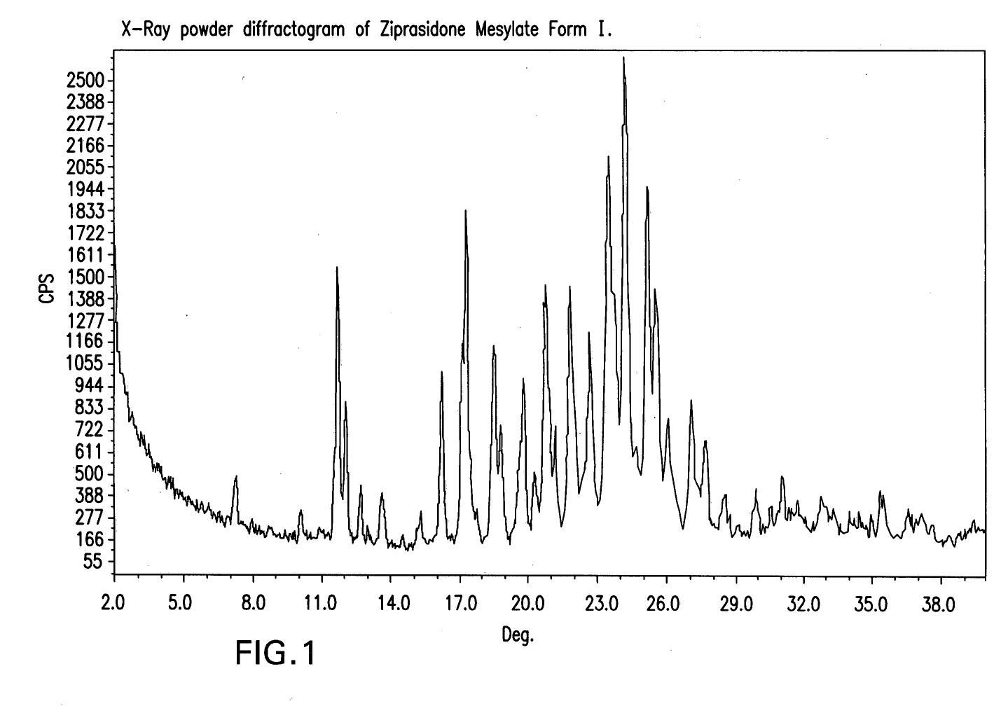

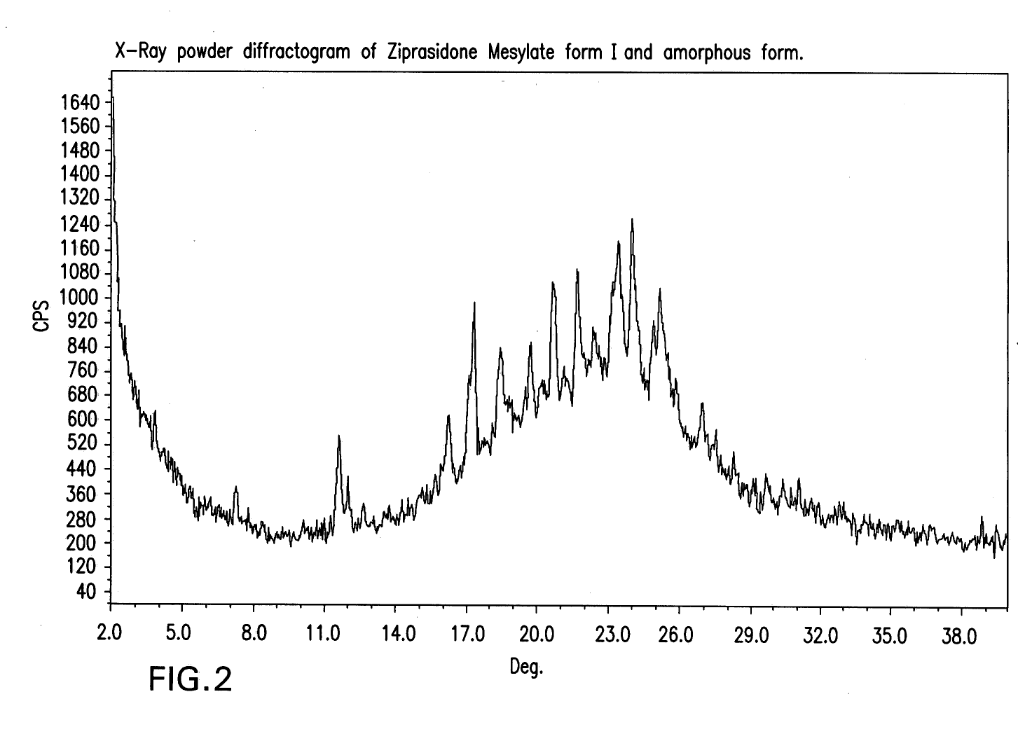

One embodiment of the invention encompasses ziprasidone mesylate form, characterized by X-ray powder diffraction peaks at about 11.7, 17.3, 23.5, 24.2, and 25.2 degrees two-theta, ±0.2 degrees two-theta. This form is denominated Form I. Form I may be characterized further by X-ray powder diffraction peaks at about 18.5, 20.7, 21.8, 22.7 and 25.7 degrees two-theta, ±0.2 degrees two-theta. Form I may be substantially identified by FIG. 1.

Form I may be an acetic acid solvate. This solvate may also be a hydrate, preferably, having about 2.3% water by weight, as measured by Karl Fisher.

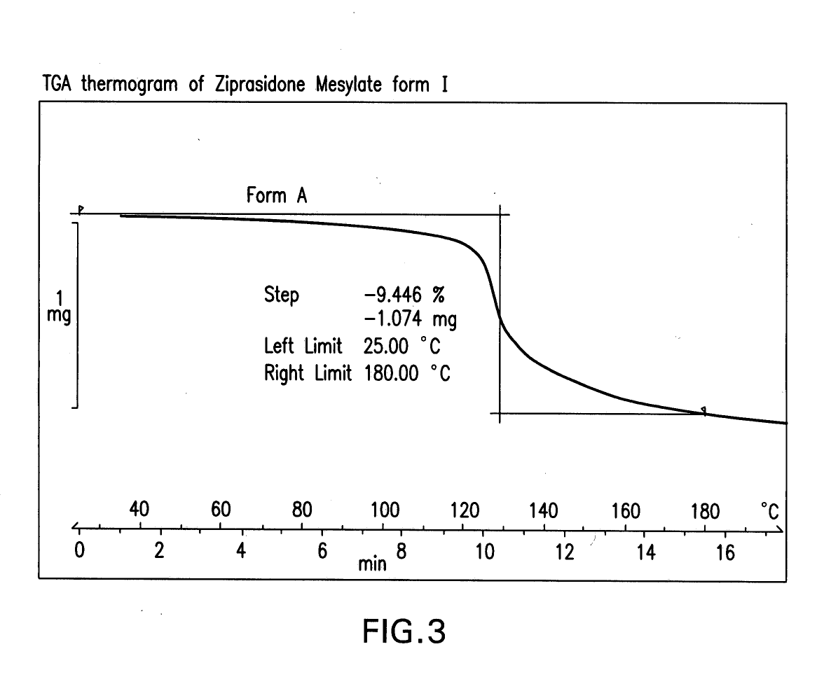

Form I has a weight loss at the range of about 25° C. to about 180° C. as measured by TGA of about 9.5% by weight, which is illustrated in FIG. 3.

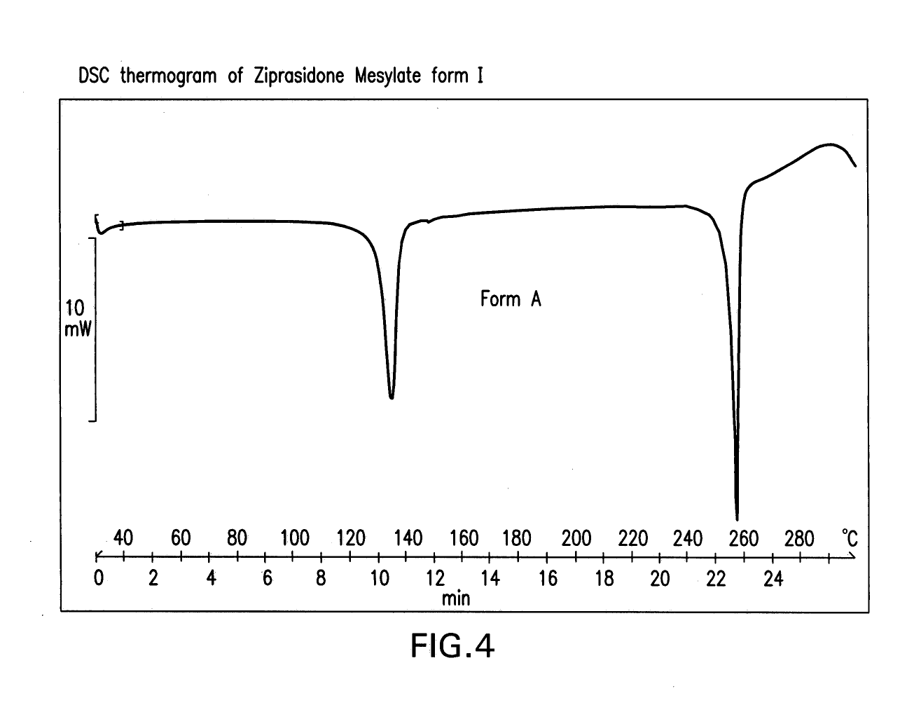

Comparison of the DSC thermogram (FIG. 4) to the TGA thermogram (FIG. 3) showed an endothermic peak (at about 134° C.) in the same range of the weight loss measured by TGA.

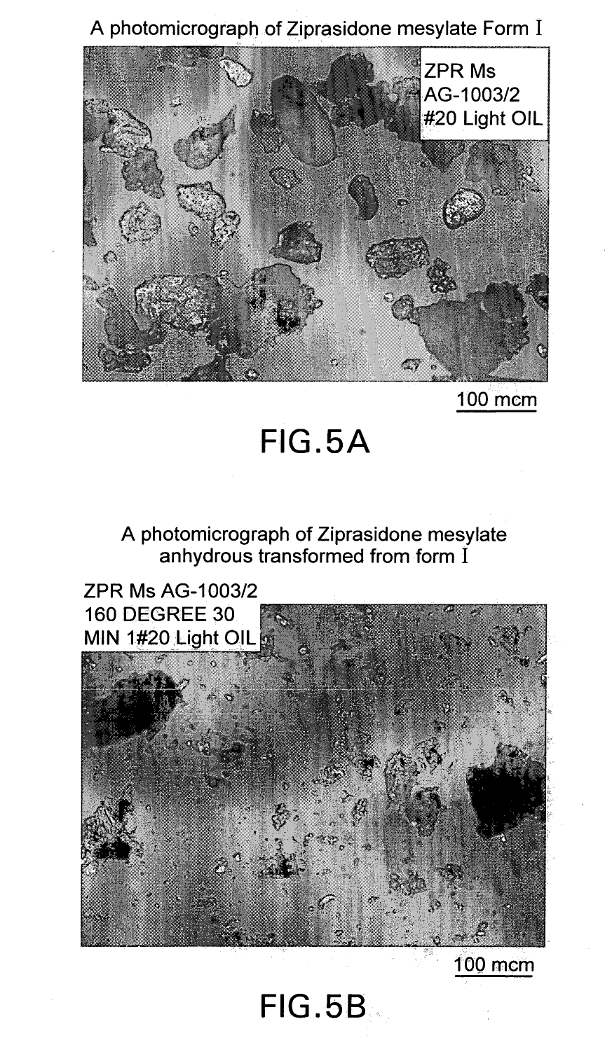

The morphology of Form I particles was found to be a tabular and equant crystals, as demonstrated in FIG. 5.

Form I was found to be stable when tested for water absorption at room temperature for 7 days under various relative humidity (“RH”) conditions, as summarized in the following table:

TABLE 1
Water Absorption of Form I
Water content

| % RH | water content (by Karl Fisher) | crystal form (by XRD) |
|---|---|---|
| 60 | 2.5 | Form I |
| 80 | 3.1 | Form I |
| 100 | 4.6 | Form I |
| Initial | 2.3 | Form I |

Another embodiment of the invention encompasses a process for crystallizing Form I from a mixture of methanesulfonic acid and a solution of ziprasidone base, acetic acid and an anti solvent selected from the group consisting of: ethanol, isopropyl alcohol, methyl-isobutyl ketone and iso-butylacetate, at a temperature of from about room temperature to about 40⁰ C.

Preferably, the acetic acid used is in a ratio of about 1:1 to about 3:1 by volume of anti solvent used, more preferably about 1.6:1 acetic acid/anti solvent.

Preferably, the total amount of solvents used is in a ratio of about 6 to 12 by volume of ziprasidone base used.

Preferably, the anti solvent is ethanol.

Preferably, the reaction occurs while stirring at about room temperature for at least about 30 minutes, more preferably, for about 2 hours.

Preferably, the mixture is cooled to about 20⁰ C, to obtain a precipitate.

Ziprasidone mesylate Form I may then be recovered by any method known in art, such as filtration and drying the precipitate, preferably at about 40° C.-70° C. at a pressure below about 100 mmHg in a vacuum oven.

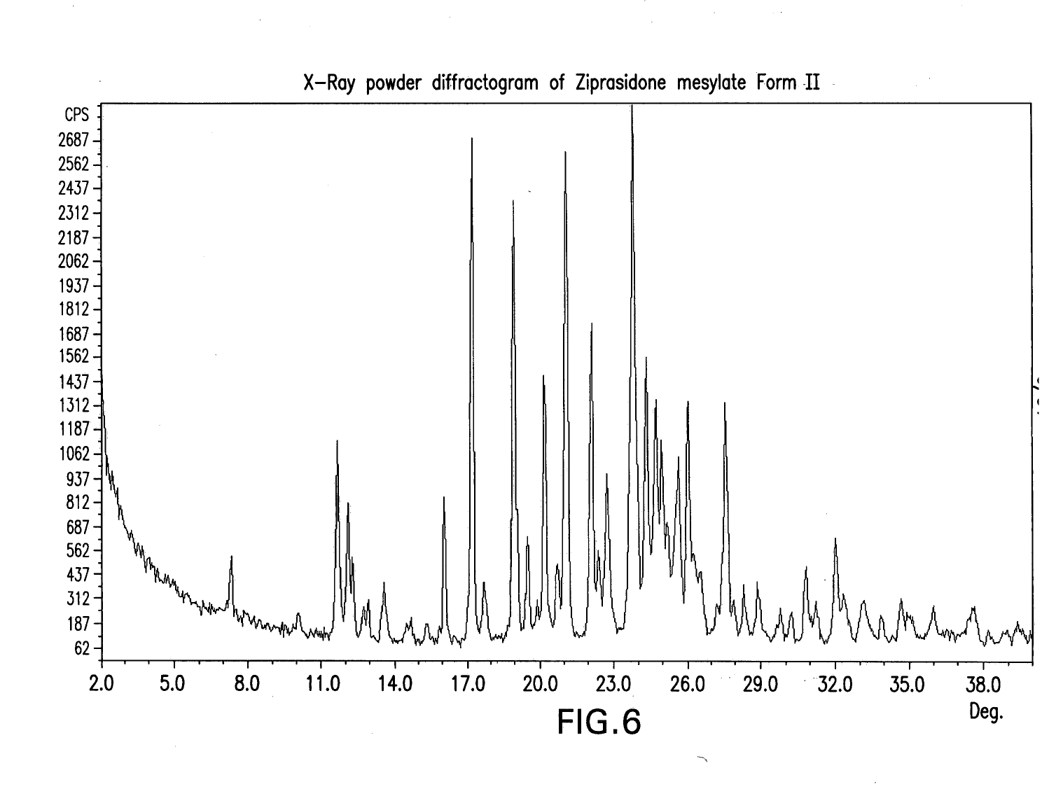

Another embodiment of the invention encompasses ziprasidone mesylate form, characterized by X-ray powder diffraction peaks at about 17.1, 18.8, 21.0 and 23.7 degrees two-theta, ±0.2 degrees two-theta. This form is denominated Form II. Form II may be characterized further by X-ray powder diffraction peaks at about 11.6, 20.1, 22.1, 24.2 and 27.5 degrees two-theta, ±0.2 degrees two-theta. Form II may be substantially identified by FIG. 6.

Form II may be an ethanol solvate. This solvate may also be a hydrate, preferably, having about 1.7% water by weight, as measured by Karl Fisher.

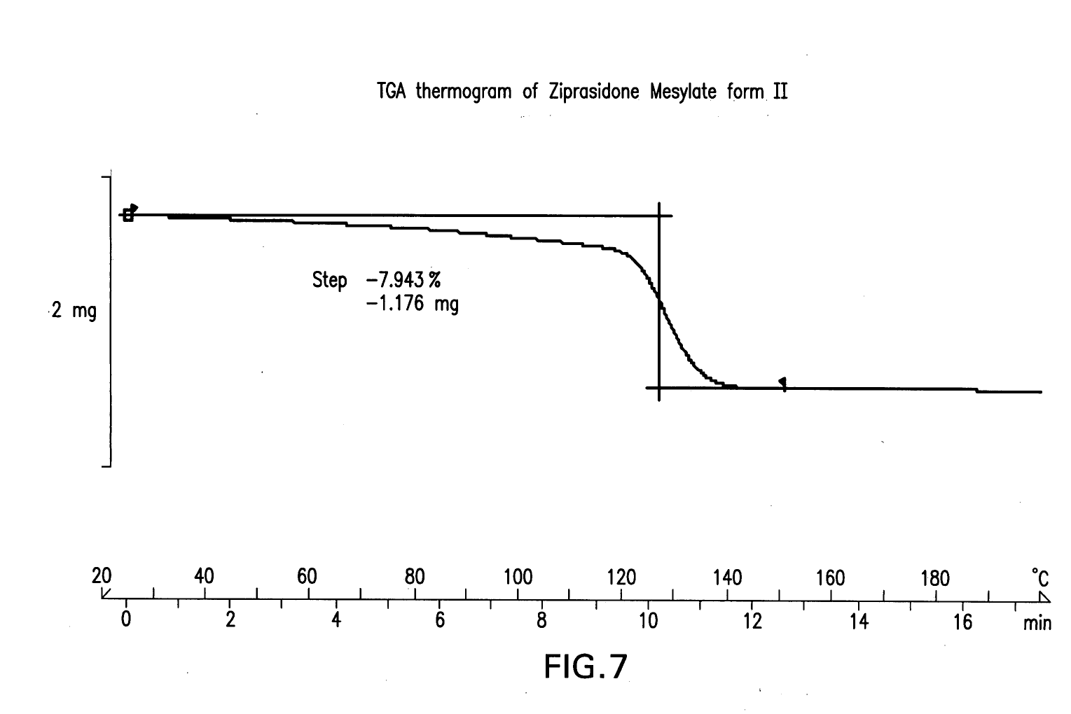

Form II has a weight loss at the range of about 25° C. to about 150° C. as measured by TGA of about 7.9% by weight, which is illustrated in FIG. 7.

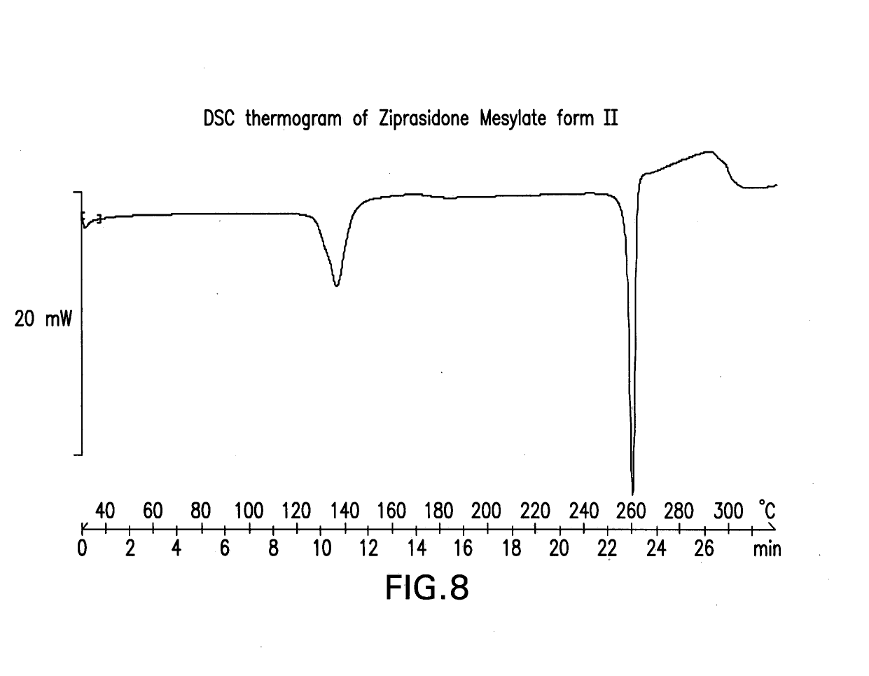

Comparison of the DSC thermogram (FIG. 8) to the TGA thermogram (FIG. 7) showed an endothermic peak (at about 130° C.) in the same range of the weight loss measured by TGA.

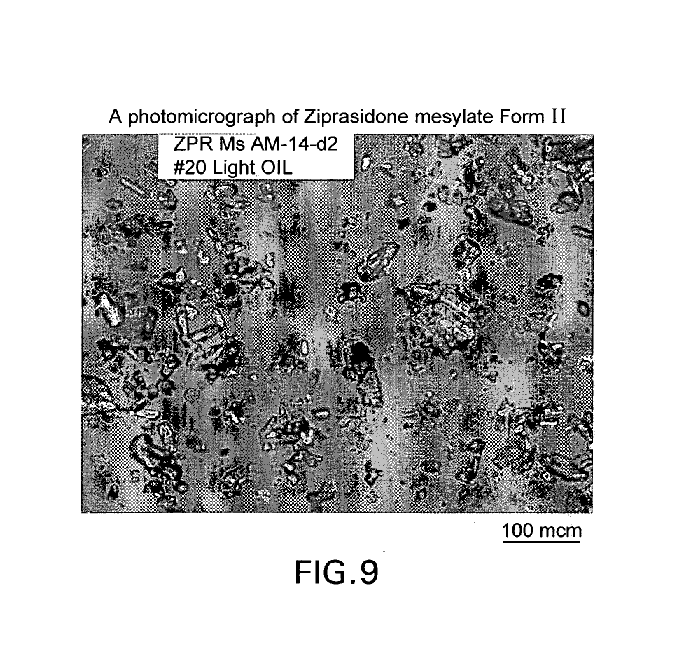

The morphology of Form II particles is demonstrated in FIG. 9.

Another embodiment of the invention encompasses a process for preparing Form II comprising: providing a slurry of ziprasidone base and ethanol, heating the slurry to a temperature of from about 40⁰ C to about 60⁰ C; combining the slurry with methanesulfonic acid; heating the mixture to a temperature of from about 60⁰ C to about 80⁰ C and cooling the mixture to about room temperature to obtain Ziprasidone mesylate Form II.

Preferably, the ethanol used is in a ratio of above about 95% by volume to water, more preferably absolute ethanol is used.

Preferably, the slurry is heated to a temperature of about 50° C.

Preferably, the mixture is heated to a temperature of about 65° C.

Preferably, the mixture is maintained, while stirring, for at least 30 minutes.

Ziprasidone mesylate Form II may then be recovered by any method known in art, such as filtration and drying the precipitate, preferably at about 40° C.-50° C. at a pressure below about 100 mmHg in a vacuum oven.

Another embodiment of the invention encompasses a process for crystallizing Form II from a mixture of methanesulfonic acid and a slurry of ziprasidone base and ethanol.

Preferably, the ethanol used is in a ratio of about 95% to about 99.9% by volume to water.

Preferably, the mixture is maintained, while stirring, at a temperature of about 5⁰ C, for about 2 hours, and then allowed to reach room temperature, to obtain Form II.

Ziprasidone mesylate Form II may then be recovered by any method known in art, such as filtration and drying the precipitate, preferably at about 40⁰ C-50⁰ C at a pressure below about 100 mmHg in a vacuum oven.

Another embodiment of the invention encompasses a process for crystallizing Form II from a mixture of methanesulfonic acid and a solution of ziprasidone base, acetic acid and ethanol at a temperature of from about 0⁰ C to about 15⁰ C.

Preferably, the acetic acid used is in a ratio of about 1:1.5 to about 3:1 by volume of ethanol used, more preferably about 1.6:1 acetic acid/ethanol.

Preferably, the total amount of acetic acid and ethanol used is in a ratio of about 6 to 12 by volume of ziprasidone base used.

Preferably, the reaction occurs while stirring at about 5⁰ C for at least 30 minutes, more preferably, for about 2 hours.

Ziprasidone mesylate Form II may then be recovered by any method known in art, such as filtration and drying the precipitate, preferably at about 40⁰ C-50⁰ C at a pressure below about 100 mmHg in a vacuum oven.

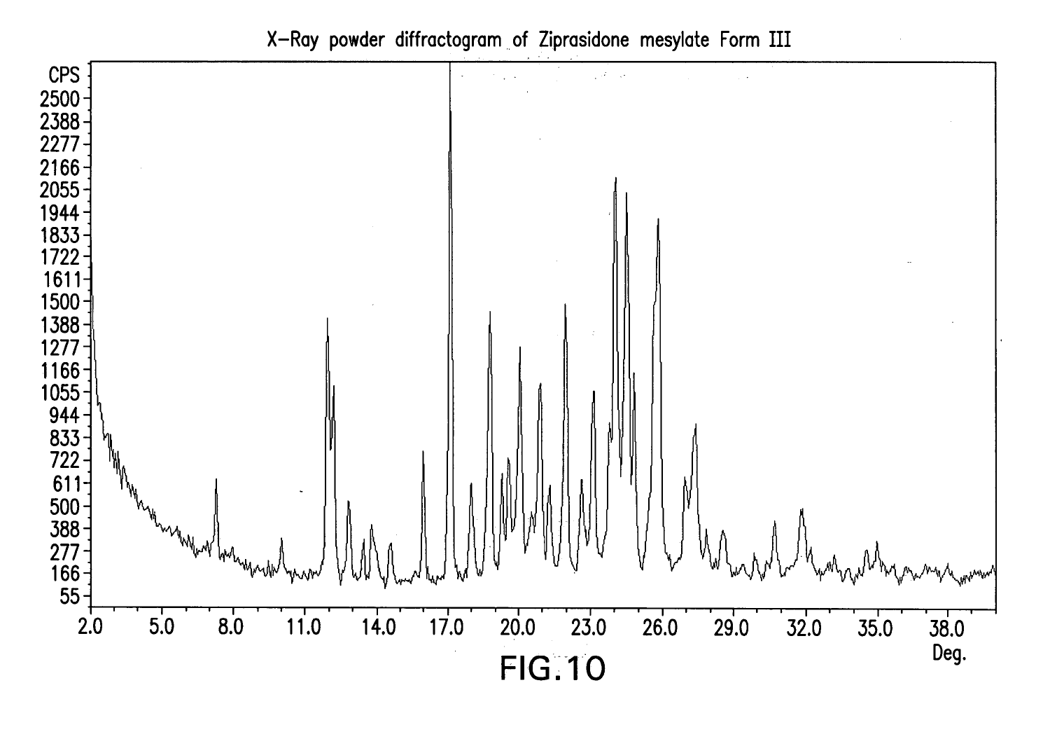

Another embodiment of the invention encompasses ziprasidone mesylate form, characterized by X-ray powder diffraction peaks at about 20.9, 21.3, 24.0, 24.5 and 25.8 degrees two-theta, ±0.2 degrees two-theta. This form is denominated Form III. Form III may be characterized further by X-ray powder diffraction peaks at about 12.0, 17.1, 18.8 and 20.1 degrees two-theta, ±0.2 degrees two-theta. Form III may be substantially identified by FIG. 10.

Form III may be an ethanol or methanol solvate. This solvate may also be a hydrate, preferably, having about 3.5% water by weight, as measured by Karl Fisher.

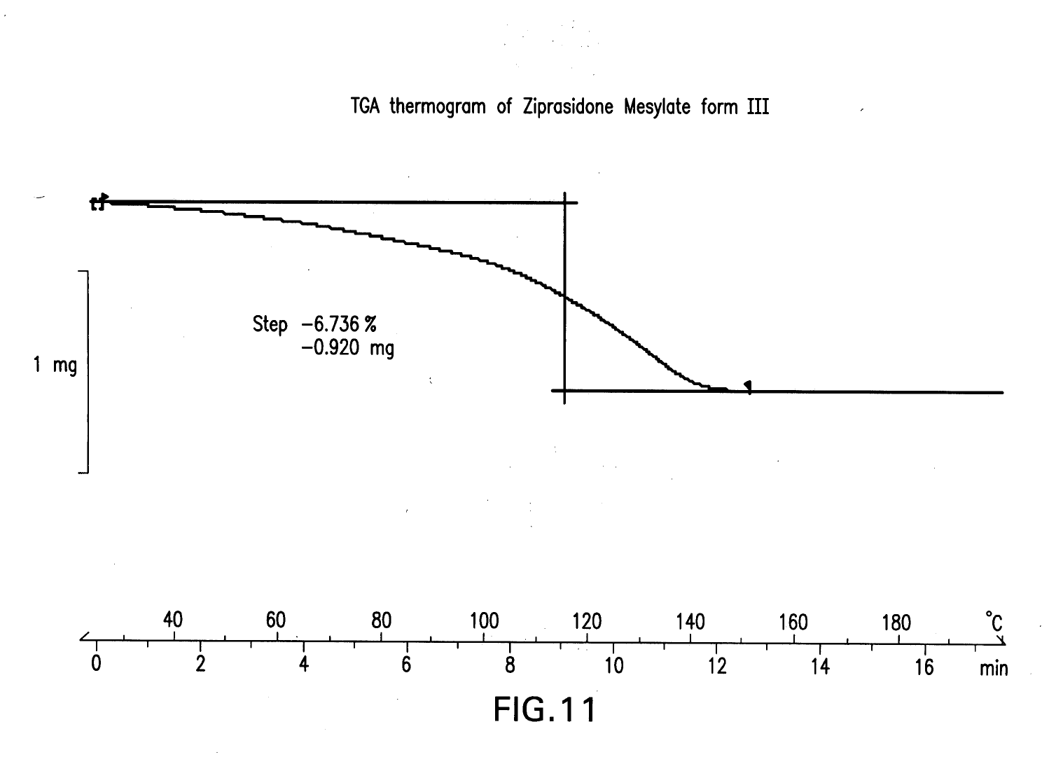

Form III has a weight loss at the range 25-150° C. as measured by TGA of about 6.7% by weight, which is illustrated in FIG. 11.

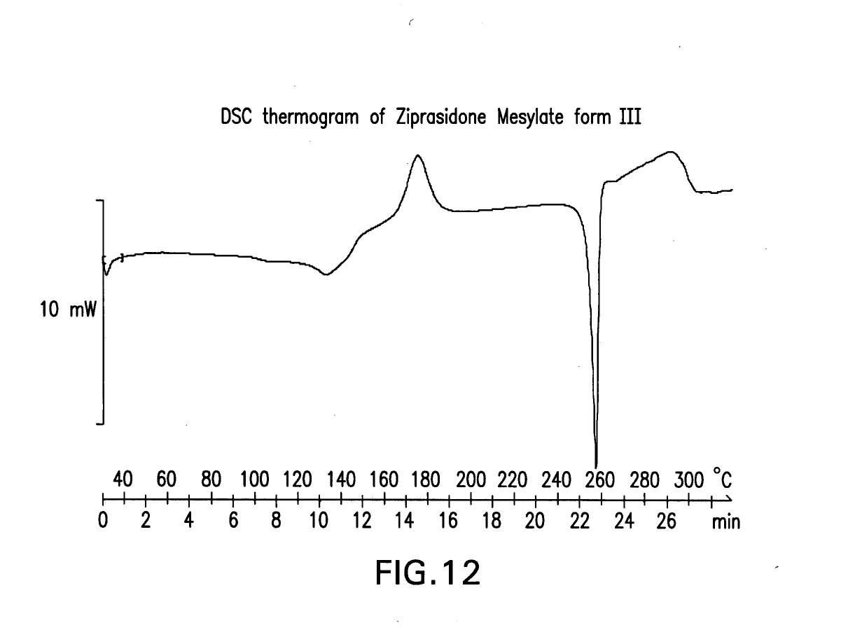

Comparison of the DSC thermogram (FIG. 12) to the TGA thermogram (FIG. 11) showed an endothermic peak (at about 120° C.-150° C.) in the same range of the weight loss measured by TGA.

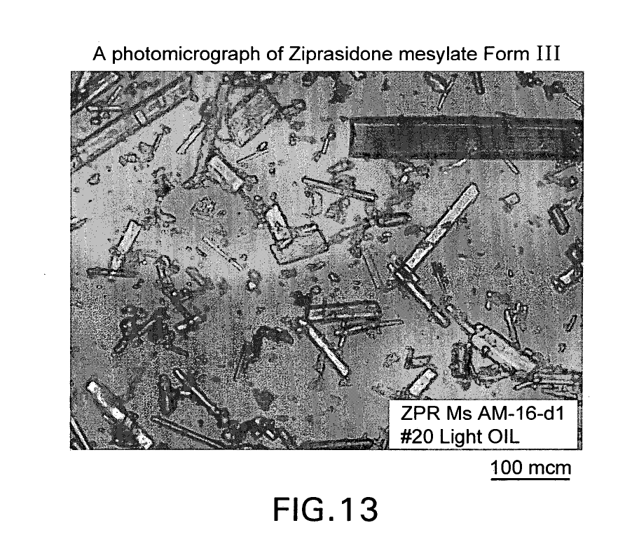

The morphology of Form III particles is demonstrated in FIG. 13.

Another embodiment of the invention encompasses a process for preparing Form III comprising: providing a mixture of ziprasidone base and a solvent selected from C₁-C₂ alcohols, heating the solution to a temperature of from about 40⁰ C to about 50⁰ C; combining the solution with methanesulfonic acid; heating the reaction mixture to a temperature of from about 60⁰ C to about 80⁰ C and cooling the reaction mixture to a temperature of from about 80⁰ C to about room temperature to obtain Ziprasidone mesylate Form III.

Preferably, the solvent is selected from a group consisting of: ethanol and methanol.

The mixture can be with or without water and with or without an acid selected from the group consisting of C₁-C₇ carboxylic acids.

Preferably, the acid is selected from a group consisting of: acetic acid and formic acid.

Preferably, whenever the mixture contains water and an acid, the ratio of acid-water-solvent is of about 1:1:2 by volume.

Preferably, the mixture is heated to a temperature of about 50° C.

Preferably, the reaction mixture is heated to a temperature of about 65° C.

Preferably, the reaction mixture is maintained, while stirring, for about 30 minutes.

Ziprasidone mesylate Form III may then be recovered by any method known in art, such as filtration and drying the precipitate, preferably at about 40° C.-60° C. at a pressure below about 100 mmHg in a vacuum oven.

Another embodiment of the invention encompasses a process for preparing Form III comprising slurring ziprasidone base, ethanol and methanesulfonic acid and to obtain Form III.

Preferably, the ethanol used is in a ratio of about 90% by volume to water.

Preferably, the reaction occurs while stirring at a temperature of about 5⁰ C for about 2 hours.

Ziprasidone mesylate Form III may then be recovered by any method known in art, such as filtration and drying the precipitate, preferably at about 40⁰ C-50⁰ C at a pressure below about 100 mmHg in a vacuum oven.

Another embodiment of the invention encompasses a process for crystallizing Forms II and III from a slurry of ziprasidone mesylate Form II and methanol.

Preferably, the reaction occurs while stirring at a temperature of about 50⁰ C for about 2 hours.

Another embodiment of the invention encompasses a process for preparing Form III by drying ziprasidone mesylate Form V.

Preferably, wet ziprasidone mesylate Form V is heated to a temperature of from about 30° C. to about 70° C., more preferably to about 45° C., for a time sufficient to obtain ziprasidone mesylate Form III. Ziprasidone mesylate Form V may be prepared as described below. As one skilled in the art will appreciate, the time required to obtain ziprasidone mesylate Form III will vary depending upon, among other factors, the amount of wet ziprasidone mesylate Form V to be dried and the drying temperature, and can be determined by taking periodic XRD's.

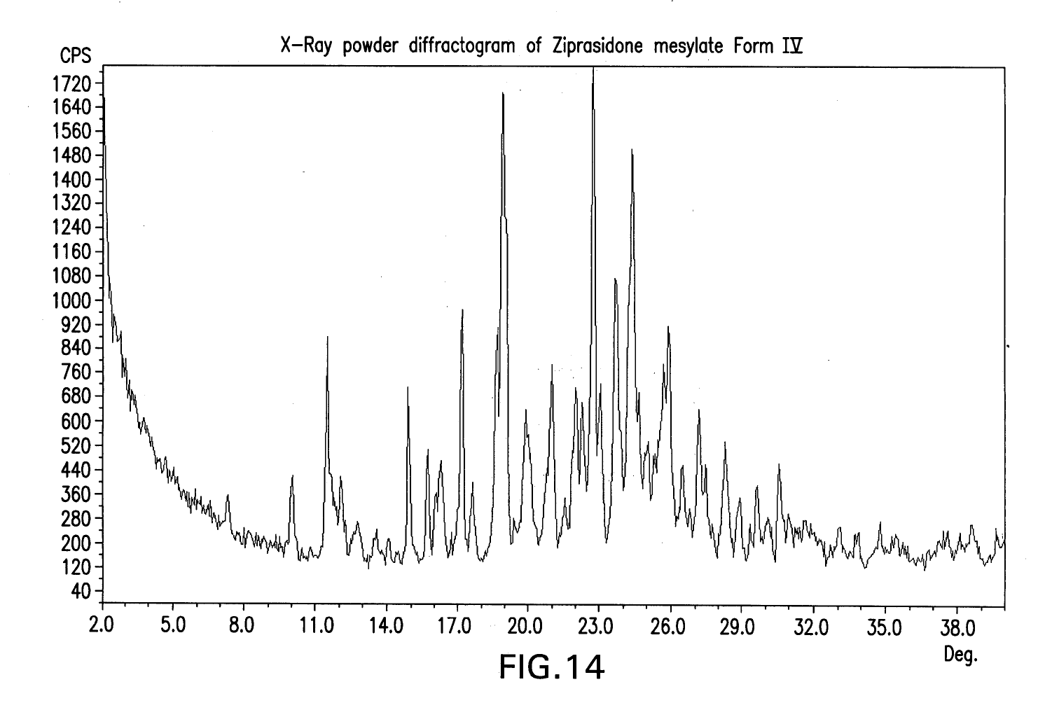

Another embodiment of the invention encompasses ziprasidone mesylate form, characterized by X-ray powder diffraction peaks at about 17.1, 18.9, 22.7, 23.6 and 24.3 degrees two-theta, ±0.2 degrees two-theta. This form is denominated Form IV. Form IV may be characterized further by X-ray powder diffraction peaks at about 11.4, 14.9 and 25.8 degrees two-theta, ±0.2 degrees two-theta. Form IV may be substantially identified by FIG. 14.

Form IV may be a methanol solvate. This solvate may also be a hydrate, preferably having about 5.4% water by weight, as measured by Karl Fisher.

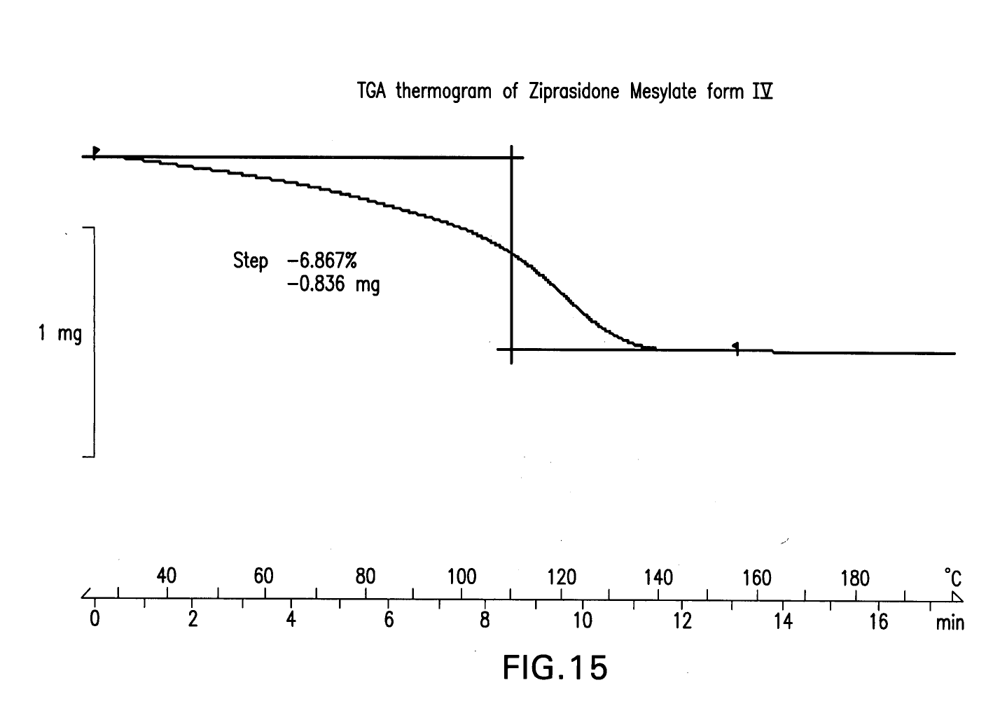

Form IV has a weight loss at the range 25-150° C. as measured by TGA of about 6.9% by weight, as illustrated in FIG. 15.

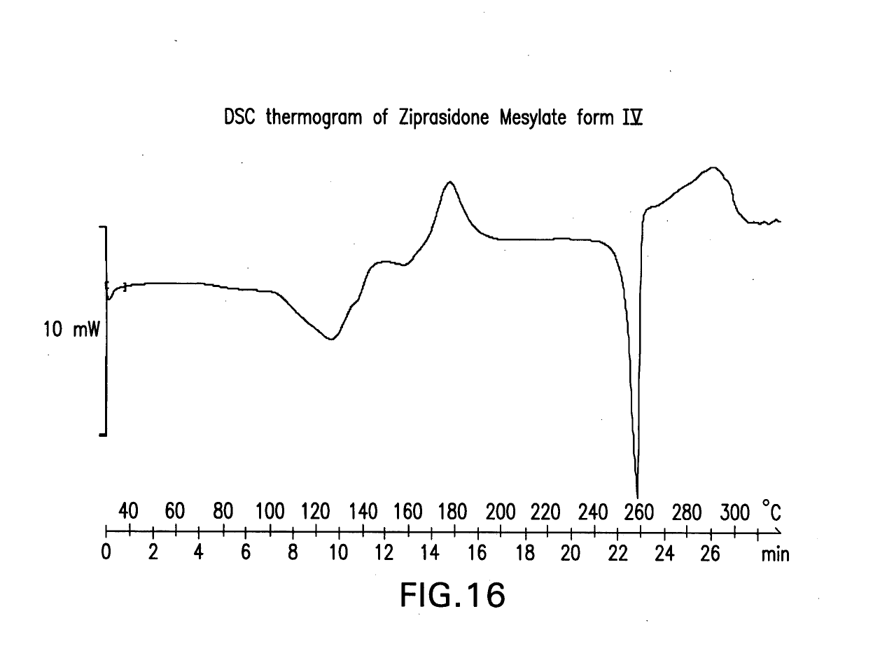

Comparison of the DSC thermogram (FIG. 16) to the TGA thermogram (FIG. 15) showed an endothermic peak (at about 100° C.-140° C.) in the same range of the weight loss measured by TGA.

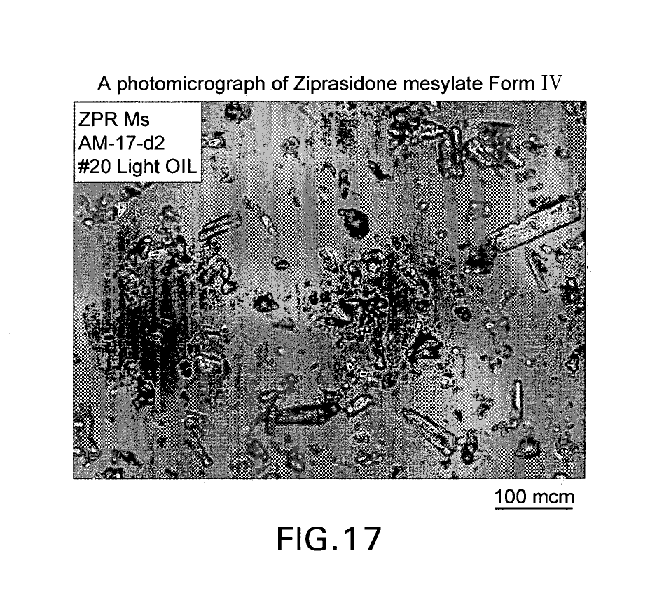

The morphology of Form IV particles is demonstrated in FIG. 17.

Another embodiment of the invention encompasses a process for preparing Form IV by drying ziprasidone mesylate Form V.

Preferably, wet ziprasidone mesylate Form V is heated to a temperature of from about 30° C. to about 70° C., more preferably to about 45° C., for a time sufficient to obtain ziprasidone mesylate Form IV. Ziprasidone mesylate Form V may be prepared as described below. As one skilled in the art will appreciate, the time required to obtain ziprasidone mesylate Form IV will vary depending upon, among other factors, the amount of wet ziprasidone mesylate Form V to be dried and the drying temperature, and can be determined by taking periodic XRD's.

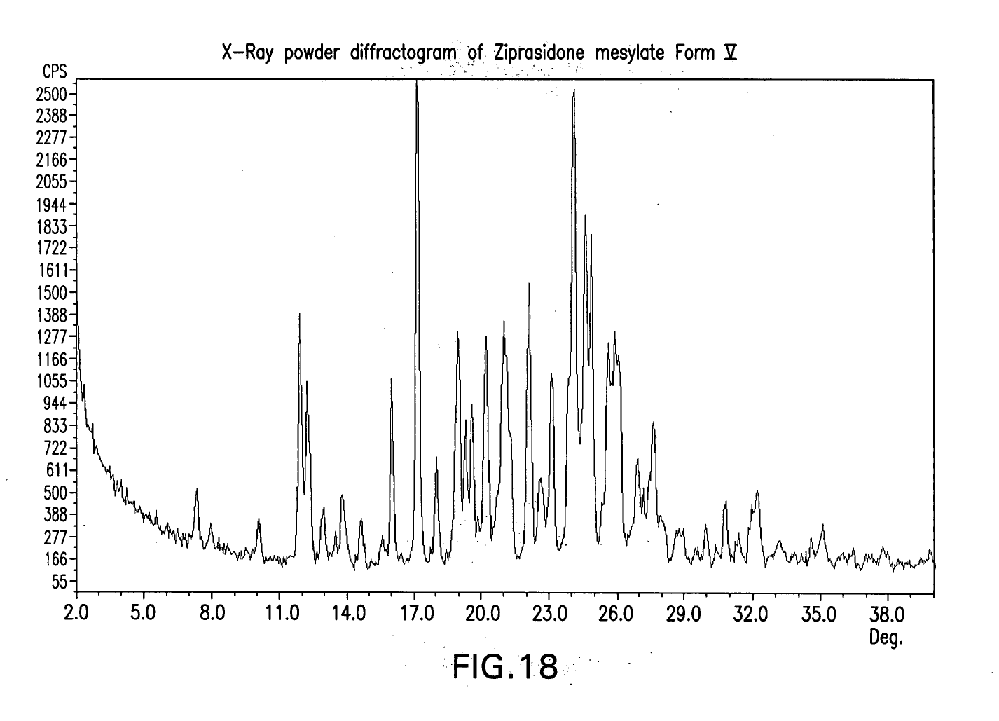

Another embodiment of the invention encompasses ziprasidone mesylate form, characterized by X-ray powder diffraction peaks at about 22.1, 25.5, 26.8, 27.1 and 27.5 degrees two-theta, ±0.2 degrees two-theta. This form is denominated Form V. Form V may be characterized further by X-ray powder diffraction peaks at about 17.1, 18.9, 20.2, 20.9 and 24.0 degrees two-theta, ±0.2 degrees two-theta. Form V may be substantially identified by FIG. 18.

Form V may be a methanol solvate. This solvate may also be a hydrate, preferably having about 1.3% water by weight, as measured by Karl Fisher.

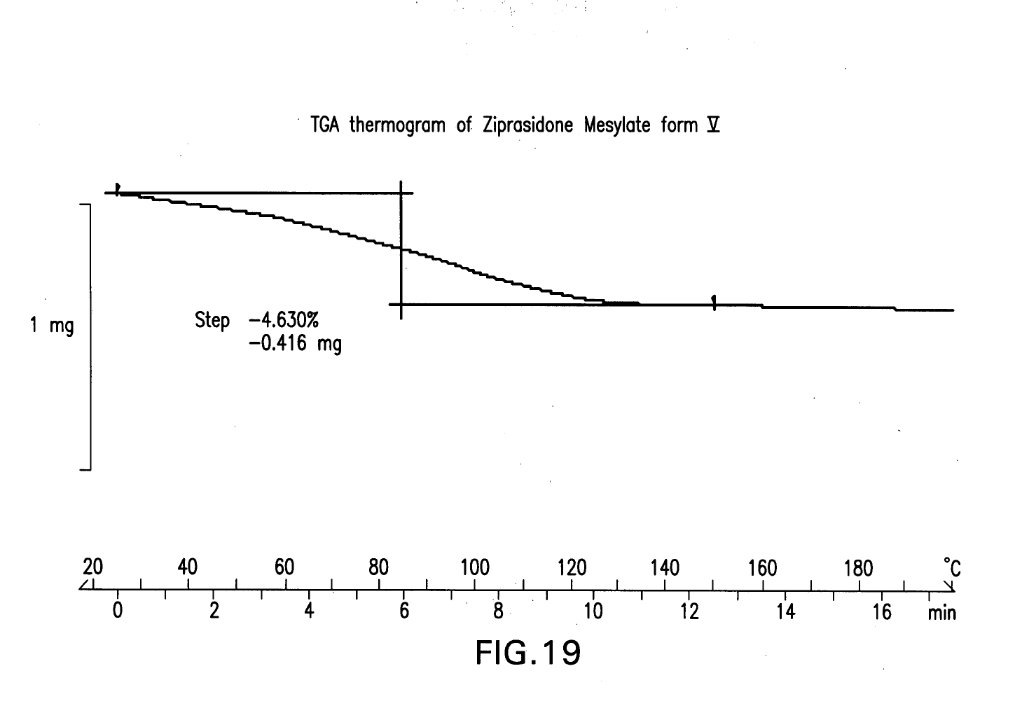

Form V has a weight loss at the range 25-150° C. as measured by TGA of about 4.6% by weight, which is illustrated in FIG. 19.

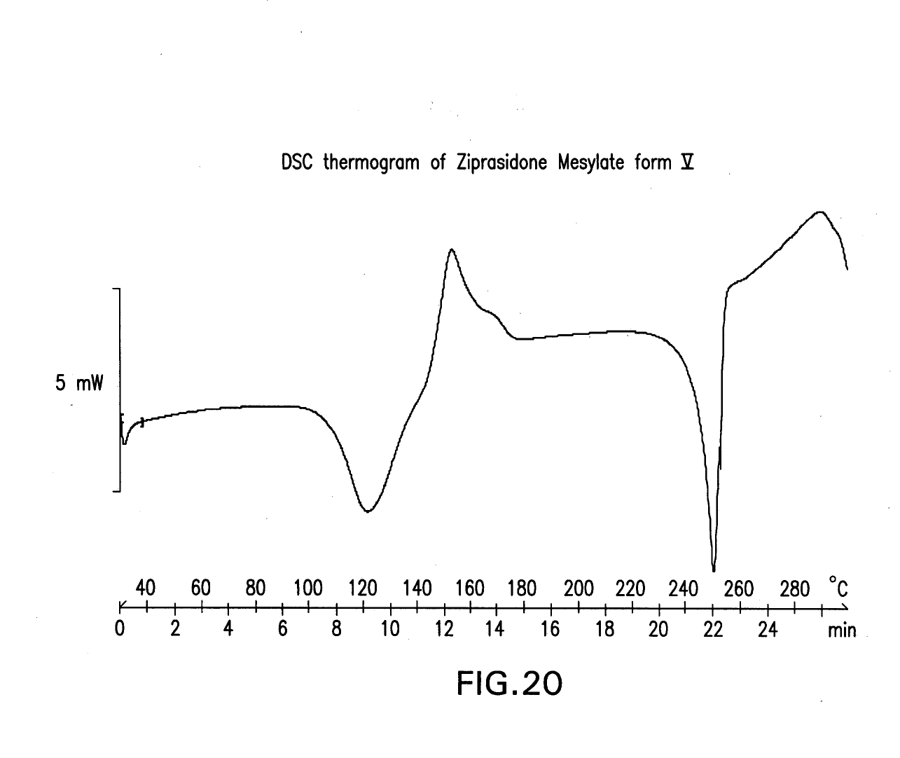

Comparison of the DSC thermogram (FIG. 20) to the TGA thermogram (FIG. 19) showed an endothermic peak (at about 100° C.-130° C.) in the same range of the weight loss measured by TGA.

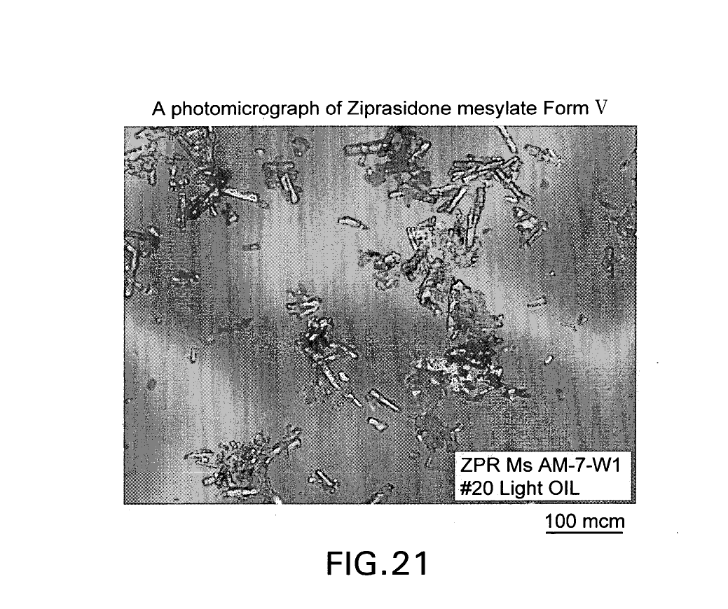

The morphology of Form V particles is demonstrated in FIG. 21.

Another embodiment of the invention encompasses a process for preparing Form V comprising: providing a slurry of ziprasidone base and methanol, heating the slurry to a temperature of from about 40⁰ C to about 60⁰ C; combining the slurry with methanesulfonic acid; heating the mixture to a temperature of from about 60⁰ C to about 80⁰ C and cooling the mixture to about room temperature to obtain Ziprasidone mesylate Form V.

Preferably, the slurry is heated to a temperature of about 50° C.

Preferably, the mixture is heated to a temperature of about 65° C.

Preferably, the mixture is maintained, while stirring, for about 30 minutes.

Preferably, after cooling to room temperature, the mixture is maintained, while stirring for about 16 hours.

Another embodiment of the invention encompasses a process for preparing Form V comprising slurring of ziprasidone base, methanol and methanesulfonic acid to obtain Form V.

Preferably, the reaction occurs while stirring at a temperature of from about 5⁰ C to about room temperature for at least 30 minutes, more preferably, for about 2 hours.

Another embodiment of the invention encompasses a process for preparing Form V comprising: providing a slurry of ziprasidone base in a mixture of a C₆ to C₉ aromatic hydrocarbon and methanol, heating the slurry to a temperature of from about 40⁰ C to about 60⁰ C; combining the slurry with methanesulfonic acid and cooling the mixture to about room temperature to obtain Ziprasidone mesylate Form V.

Preferably, the aromatic hydrocarbon is selected from the group consisting of: toluene, chlorobenzene, orto-dichlorobenzene and xylene. More preferably, the aromatic hydrocarbon is toluene.

Preferably, the ratio of toluene to methanol is from about 9:1 to about 7:1 by volume, more preferably, 8:1 by volume.

Preferably, the slurry is heated to a temperature of about 45° C.

Preferably, the mixture is maintained, while stirring, for about 1 hour.

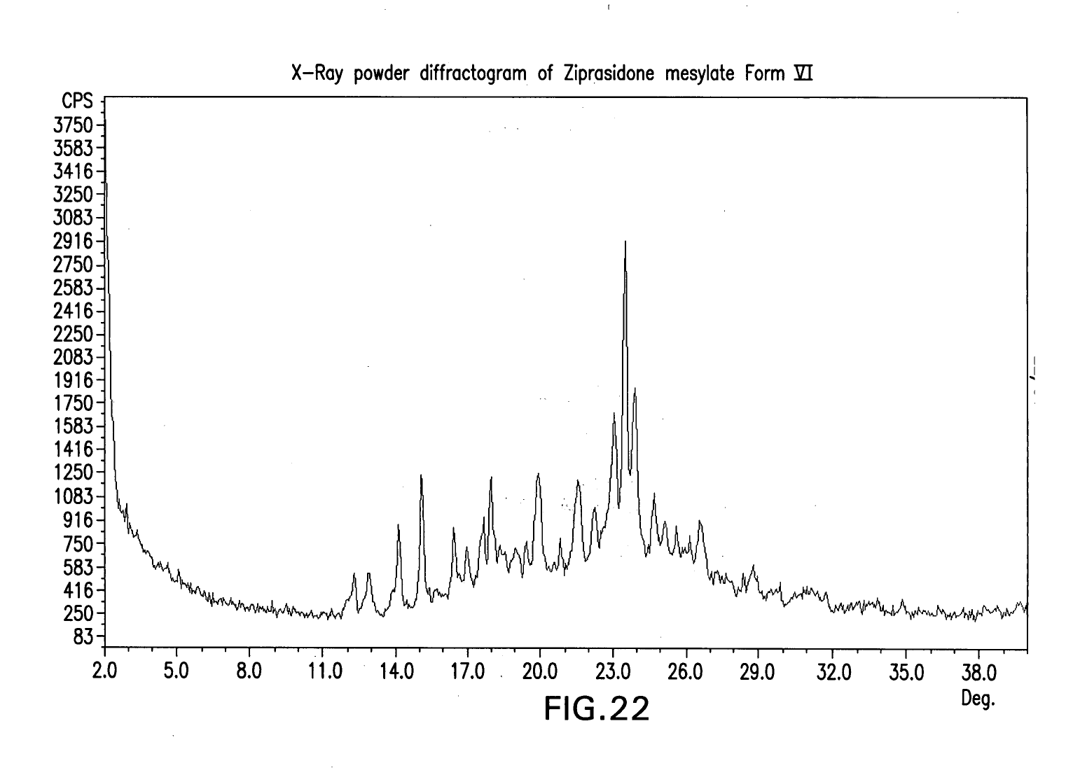

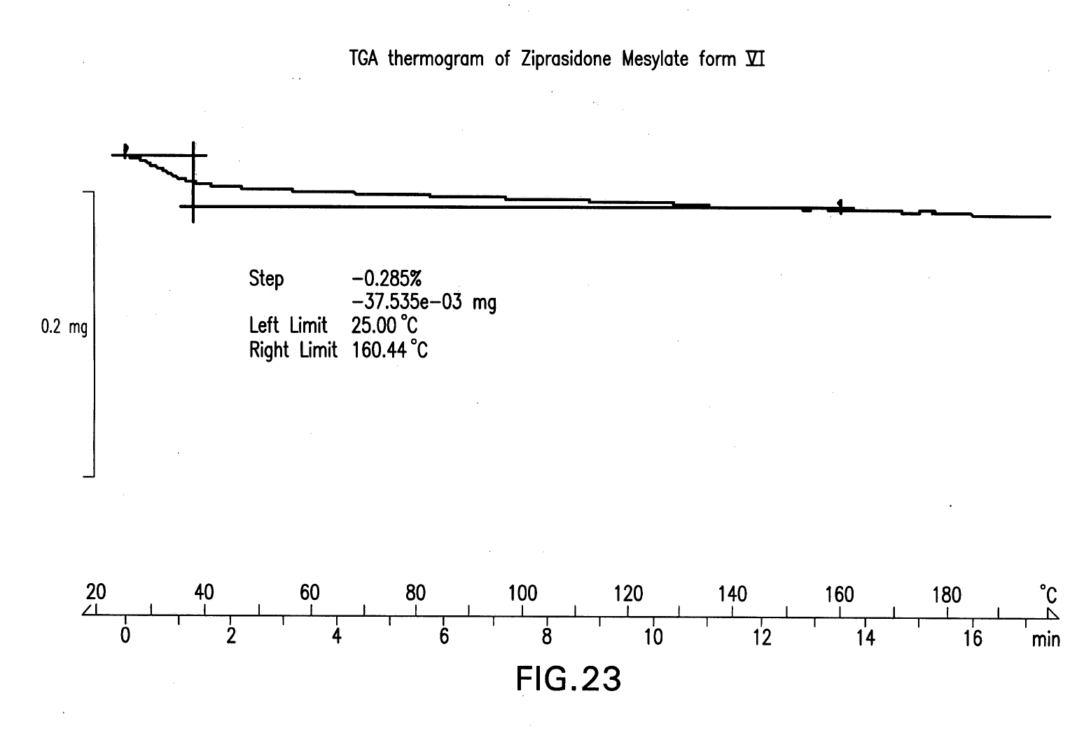

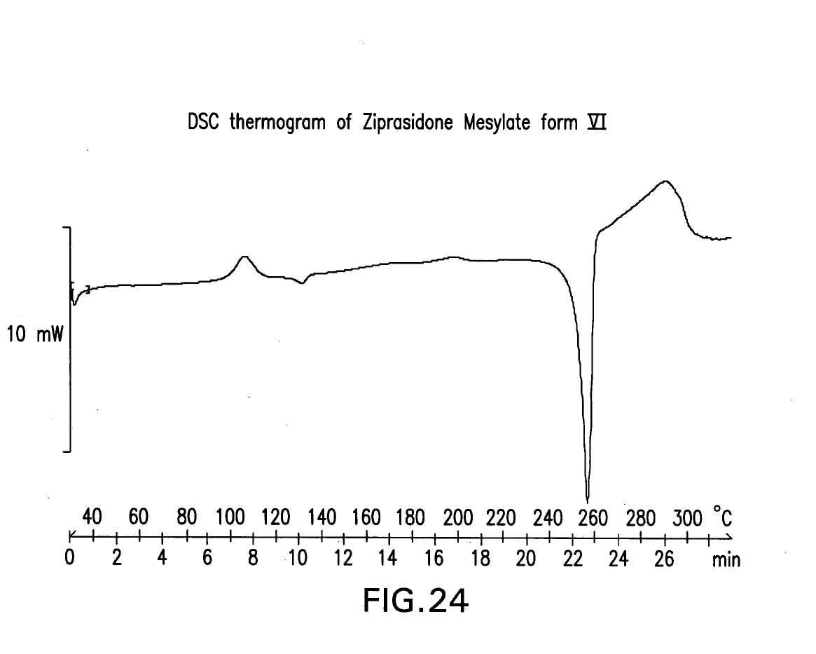

Another embodiment of the invention encompasses ziprasidone mesylate form, characterized by X-ray powder diffraction peaks at about 15.1, 23.0, 23.5 and 23.8 degrees two-theta, ±0.2 degrees two-theta. This form is denominated Form VI. Form VI may be characterized further by X-ray powder diffraction peaks at about 14.1, 17.9, 19.9, 21.6 and 24.6 degrees two-theta, ±0.2 degrees two-theta. Form VI may be substantially identified by FIG. 22.

Form VI is a monohydrate form of ziprasidone mesylate, preferably having about 0.3%-1.9% water by weight, as measured by Karl Fisher.

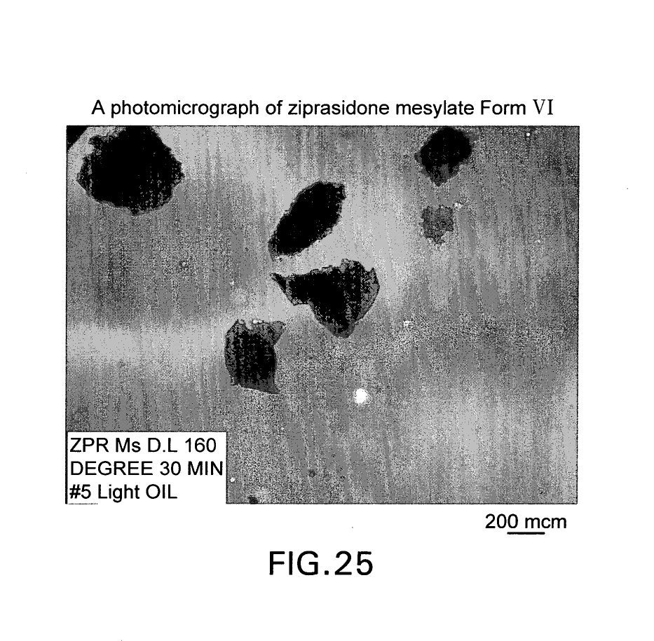

The morphology of Form VI particles is demonstrated in FIG. 25.

Another embodiment of the invention encompasses a process for preparing Form VI by heating ziprasidone mesylate dihydrate lath.

Preferably, ziprasidone mesylate dihydrate lath is heated to a temperature above 100°, more preferably to about 160° C., for a time sufficient to obtain ziprasidone mesylate Form VI. ziprasidone mesylate dihydrate lath may be prepared as described below. As one skilled in the art will appreciate, the time required to obtain ziprasidone mesylate Form VI will vary depending upon, among other factors, the drying temperature, and can be determined by taking periodic XRD's.

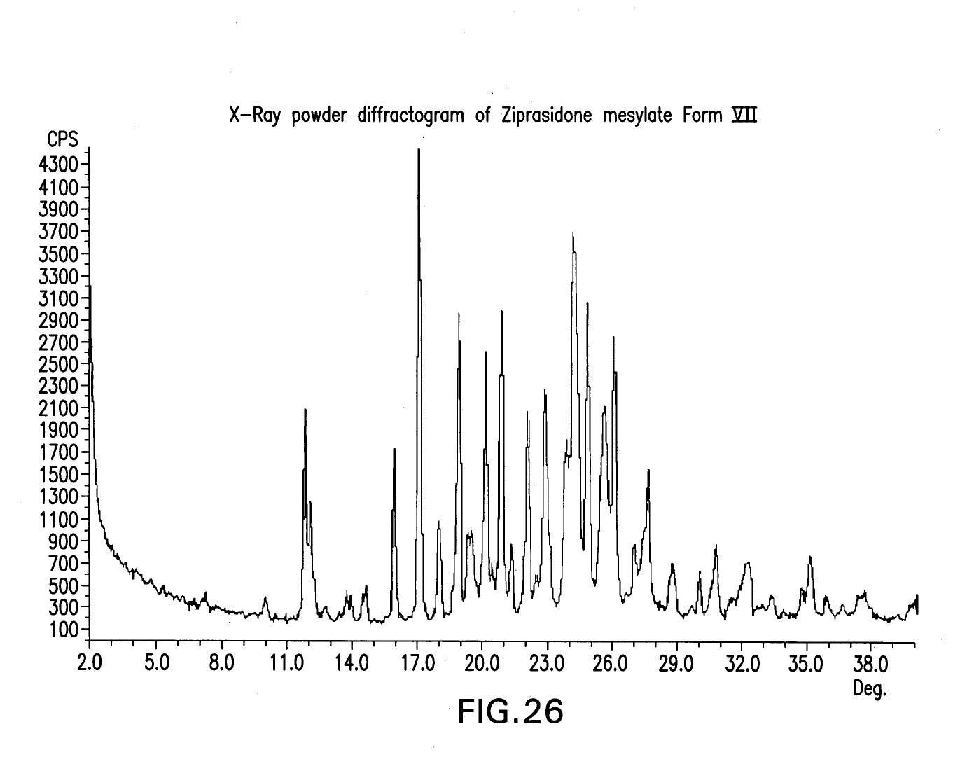

Another embodiment of the invention encompasses ziprasidone mesylate form, characterized by X-ray powder diffraction peaks at about 17.2, 19.0, 21.0, 24.3 and 24.9 degrees two-theta, ±0.2 degrees two-theta. This form is denominated Form VII. Form VII may be characterized further by X-ray powder diffraction peaks at about 11.9, 20.3, 23.0 and 26.5 degrees two-theta, ±0.2 degrees two-theta. Form VII may be substantially identified by FIG. 26.

Form VII may be a methanol solvate. This solvate may also be a hydrate, preferably having about 2.3% water by weight, as measured by Karl Fisher.

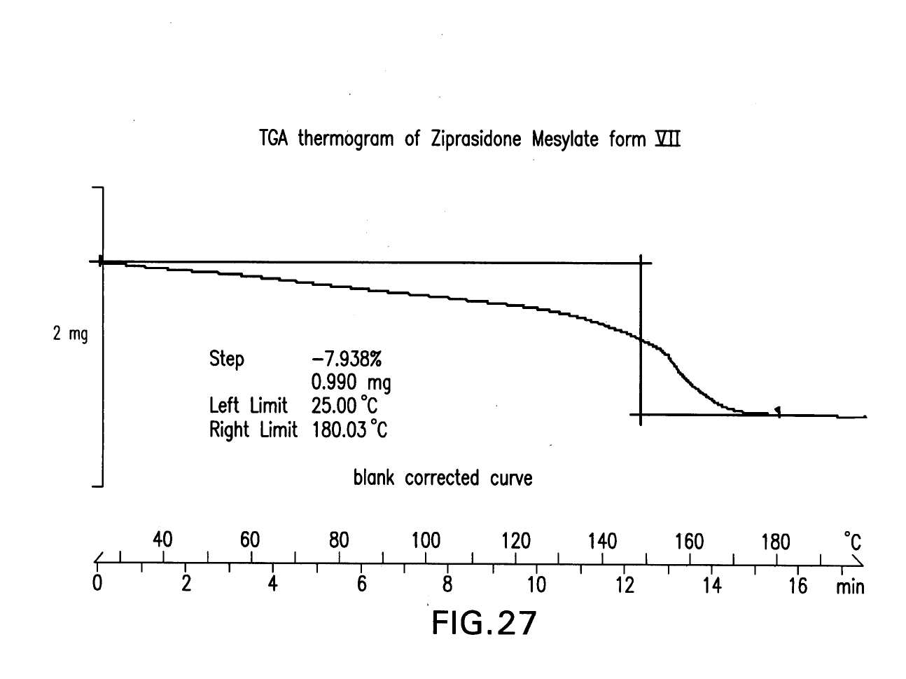

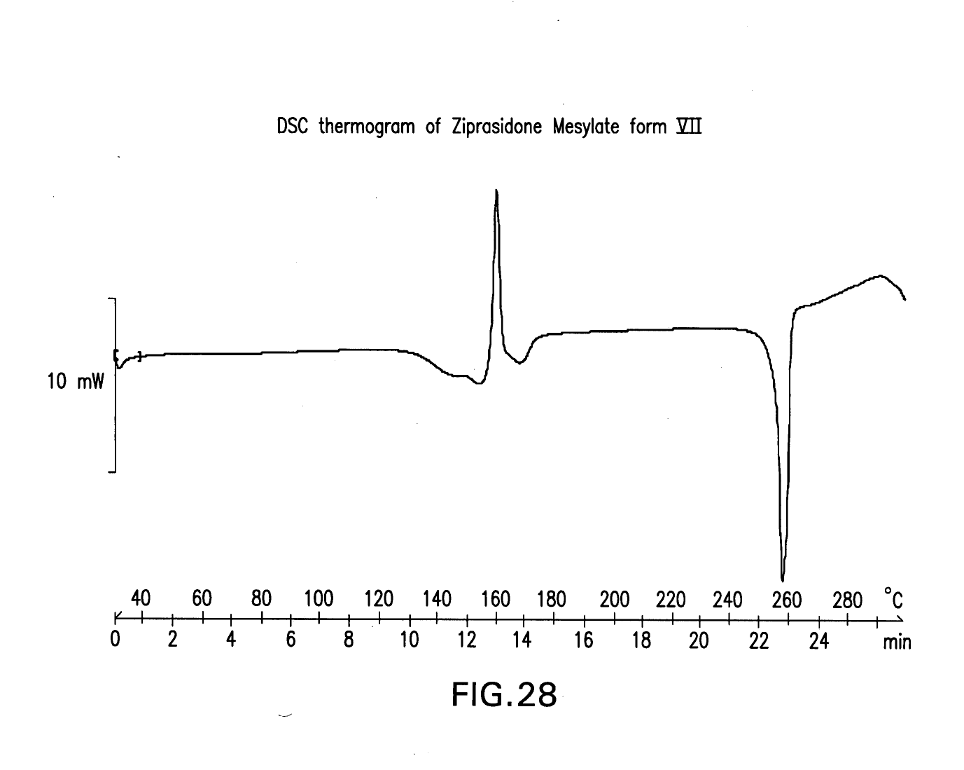

Form VII has a weight loss at the range of 25-180° C. as measured by TGA of about 7.9% by weight, which is illustrated in FIG. 27.

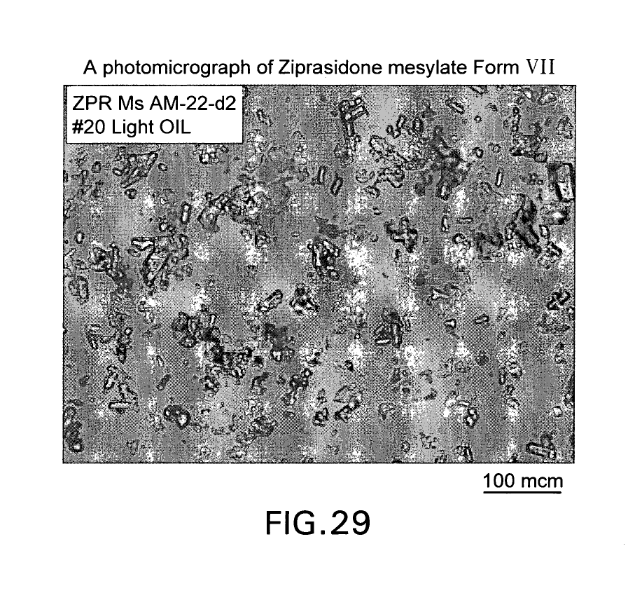

The morphology of Form VII particles is demonstrated in FIG. 29.

Another embodiment of the invention encompasses a process for crystallizing Form VII from a mixture of methanesulfonic acid and a solution of ziprasidone base, formic acid, and a solvent selected from the group consisting of C₁₋₅ alcohol and water, at a temperature of from about 5⁰ C to about room temperature.

Preferably, the solvent is formic acid and mixtures thereof with methanol and water.

Preferably, the formic acid used is in a ratio of about 1:1 to about 1:3 by volume of methanol used, more preferably about 1:2 formic acid/methanol, most preferably, 1:2:1 formic acid/methanol/water.

Preferably, the reaction occurs while stirring at a temperature of from about 5⁰ C to about room temperature for about 2 hours.

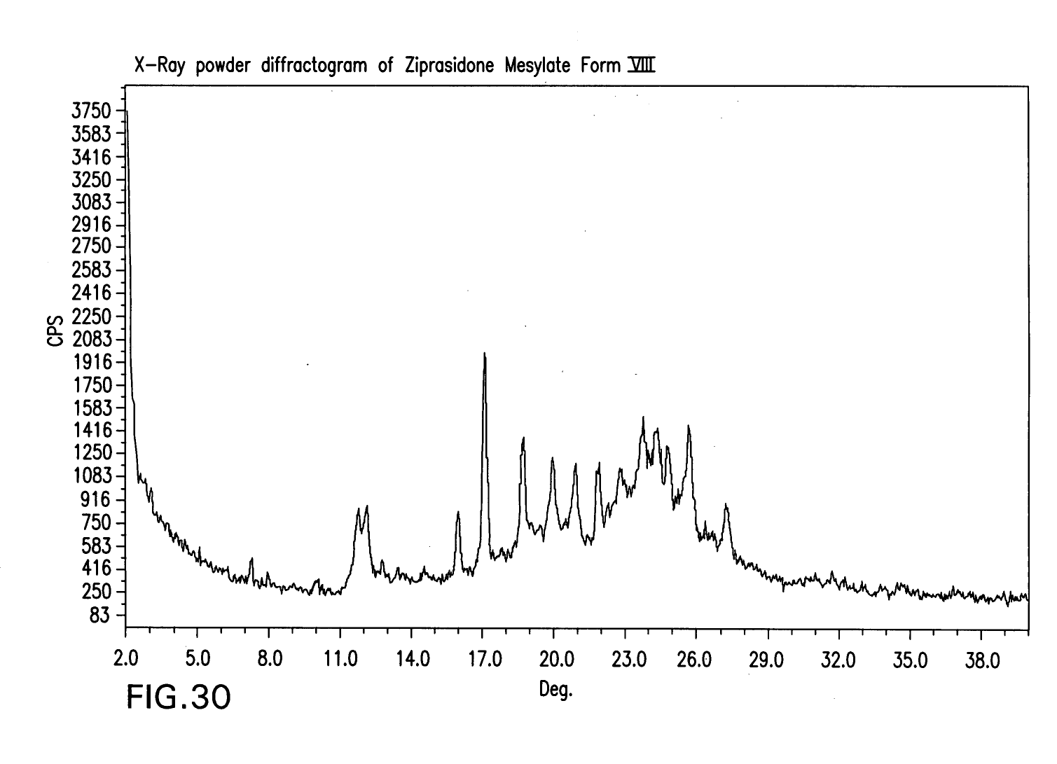

Another embodiment of the invention encompasses ziprasidone mesylate form, characterized by X-ray powder diffraction peaks at about 17.1, 18.7, 23.8 and 24.4 degrees two-theta, ±0.2 degrees two-theta. This form is denominated Form VIII. Form VIII may be characterized further by X-ray powder diffraction peaks at about 11.8, 12.1, 20.0, 20.9, 24.9 and 25.7 degrees two-theta, ±0.2 degrees two-theta. Form VIII may be substantially identified by FIG. 30.

Form VIII may be an ethanol solvate. This solvate may also be a hydrate, preferably having about 4.0% water by weight, as measured by Karl Fisher.

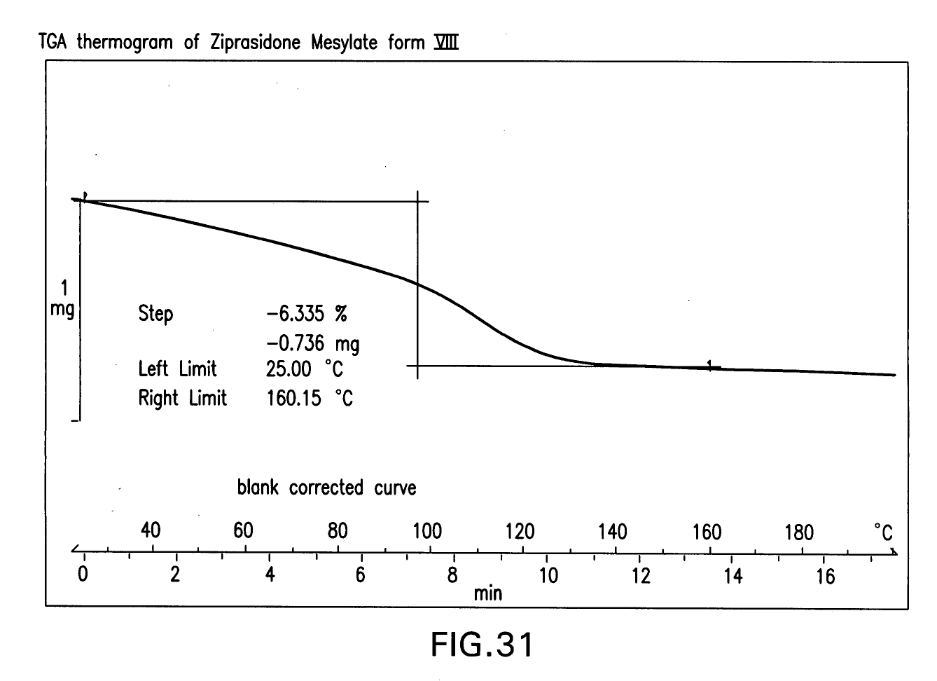

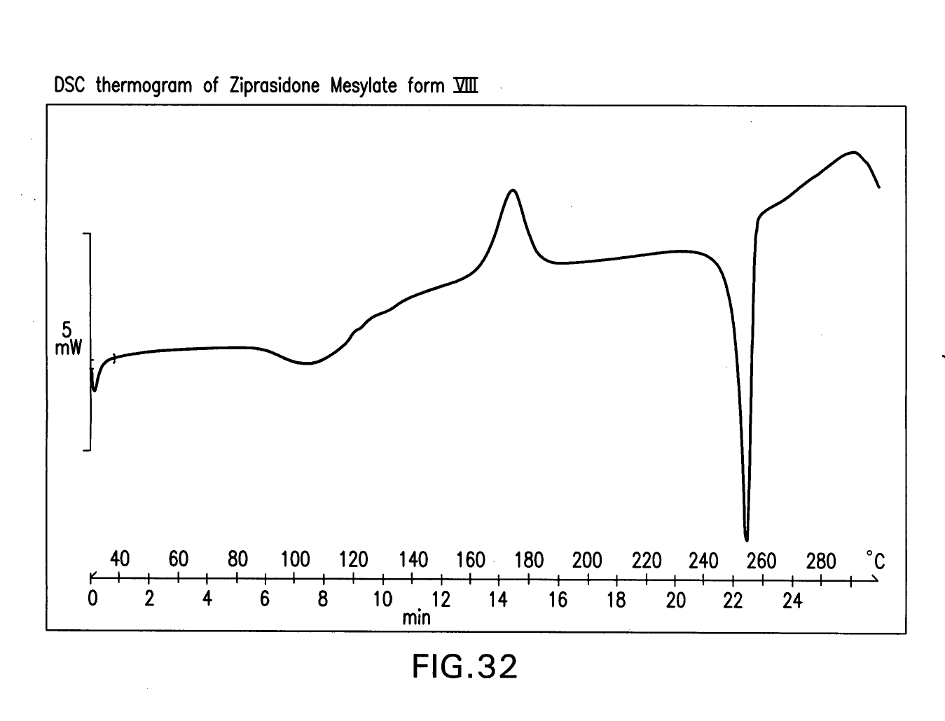

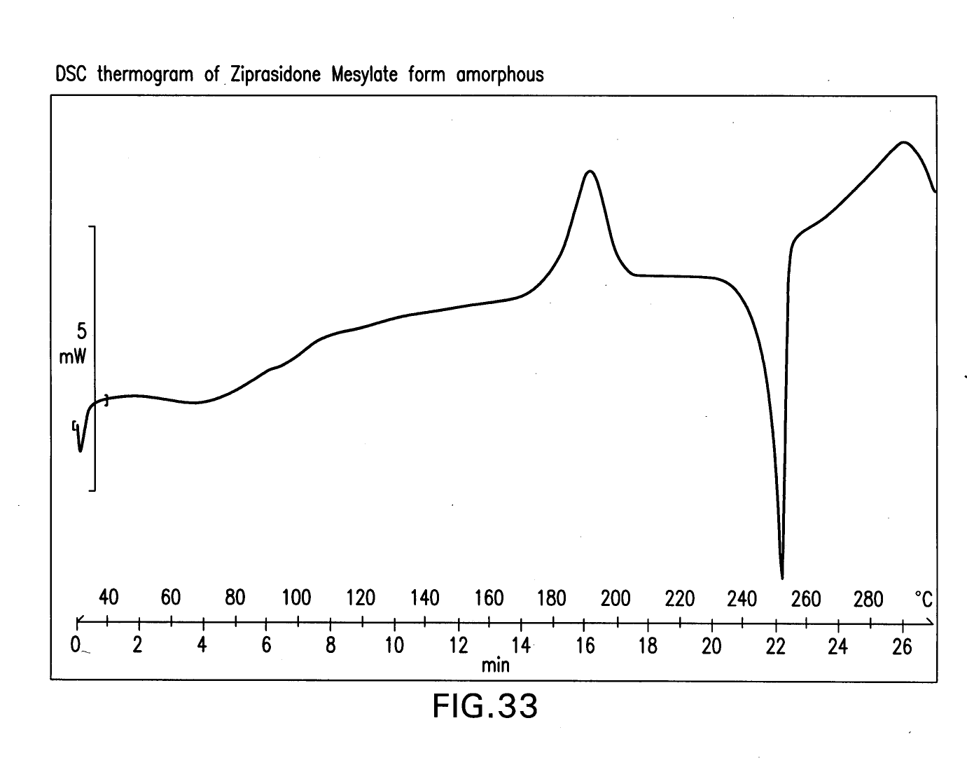

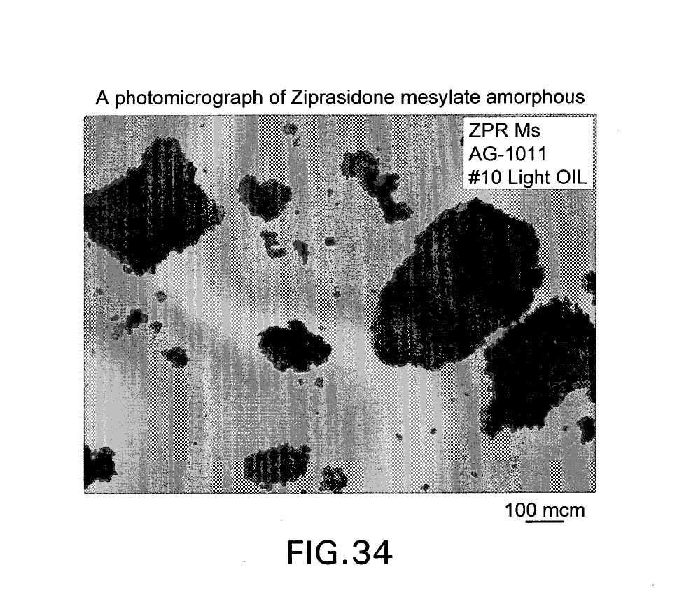

Form VIII has a weight loss at the range of 25-160° C. as measured by TGA of about 6.3% by weight, which is illustrated in FIG. 31.

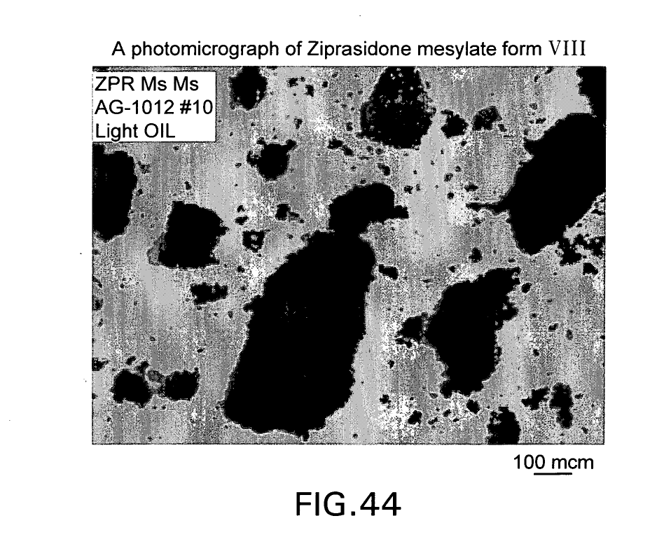

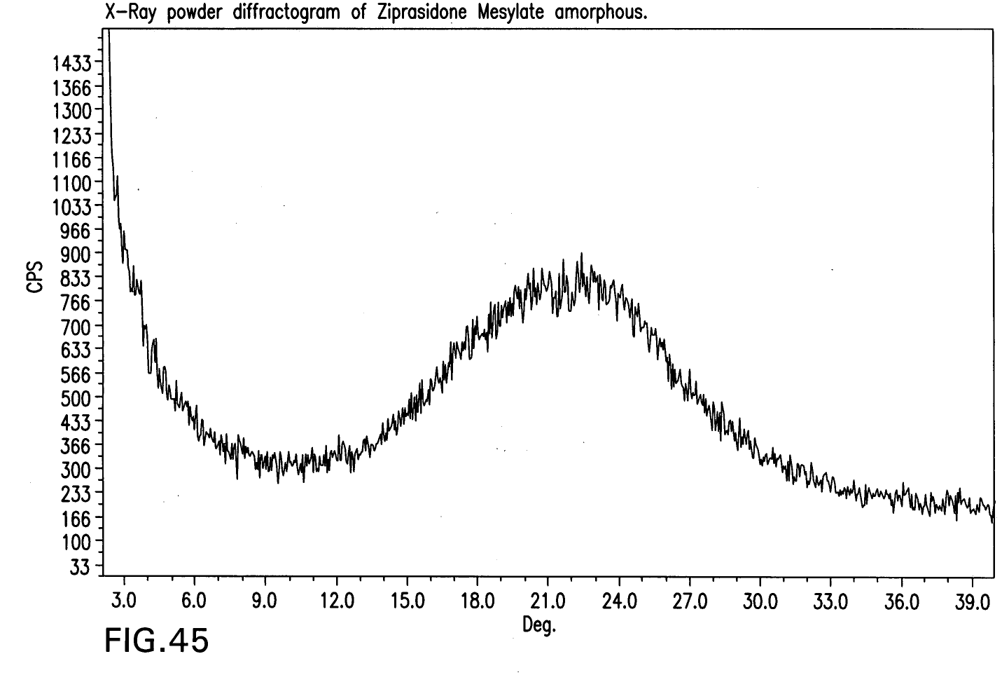

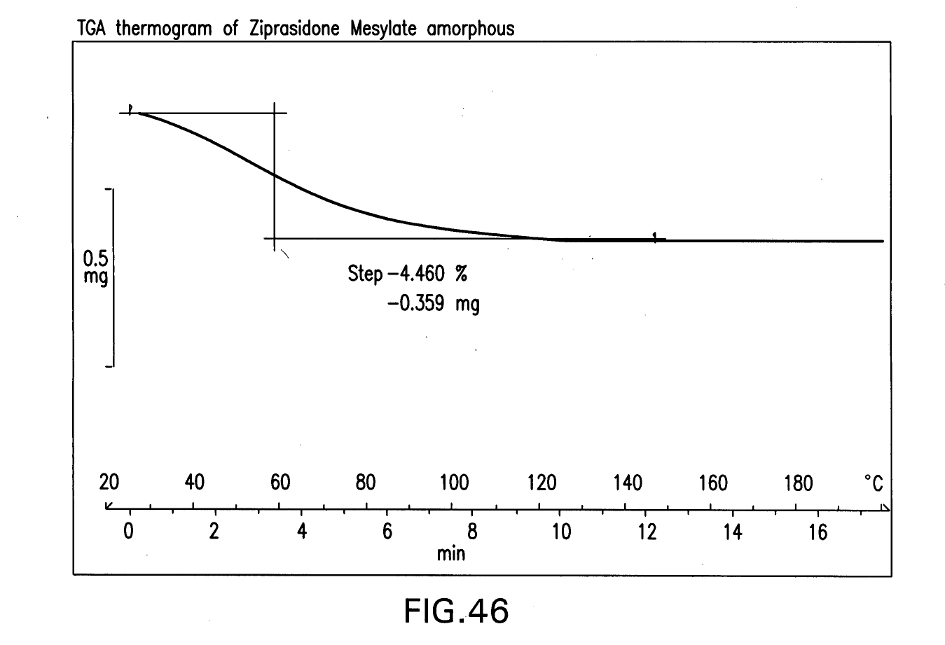

The morphology of Form VIII particles is demonstrated in FIG. 44.

Another embodiment of the invention encompasses a process for preparing Form VIII by drying ziprasidone mesylate Form IX.

Preferably, wet ziprasidone mesylate Form IX is heated to a temperature of from about 60° C. to about 100° C., more preferably to about 80° C., for less than about 20 hours. Ziprasidone mesylate Form IX may be prepared as described below.

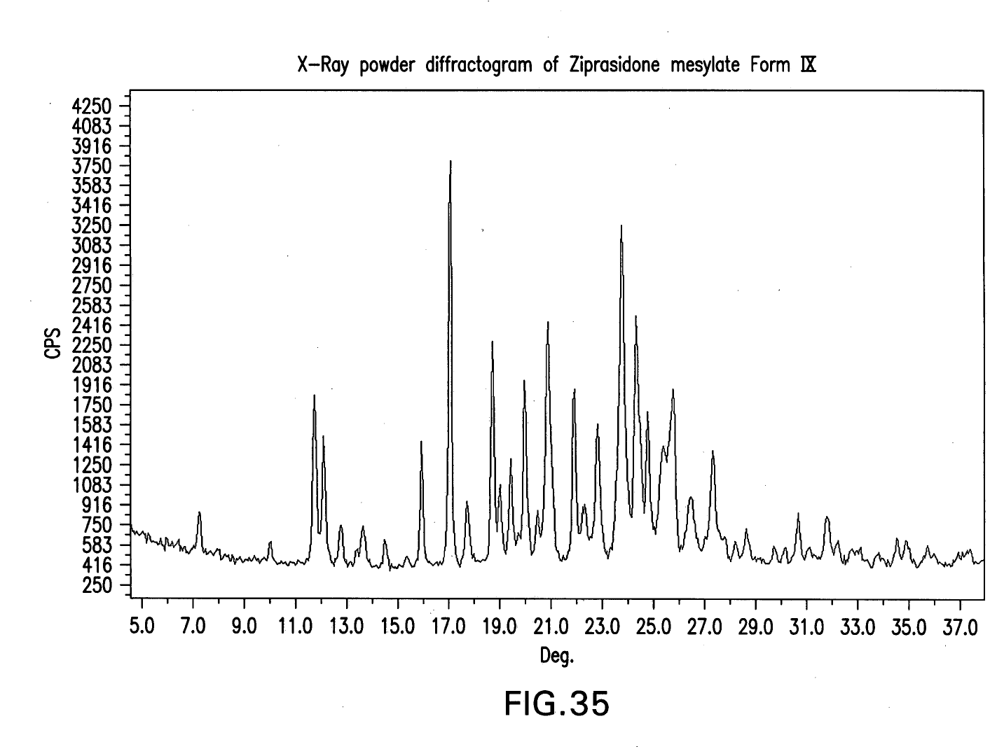

Another embodiment of the invention encompasses ziprasidone mesylate form, characterized by X-ray powder diffraction peaks at about 17.1, 18.7, 20.9, 23.8 and 24.3 degrees two-theta, ±0.2 degrees two-theta. This form is denominated Form IX. Form IX may be characterized further by X-ray powder diffraction peaks at about 11.7, 20.0, 21.0 and 25.8 degrees two-theta, ±0.2 degrees two-theta. Form IX may be substantially identified by FIG. 35.

Form IX may be an ethanol solvate. This solvate may also a hydrate, preferably, sesquihydrate, having about 4.7% water by weight, as measured by Karl Fisher.

Form IX has a weight loss at the range of 25-150° C. as measured by TGA of about 6.7% by weight.

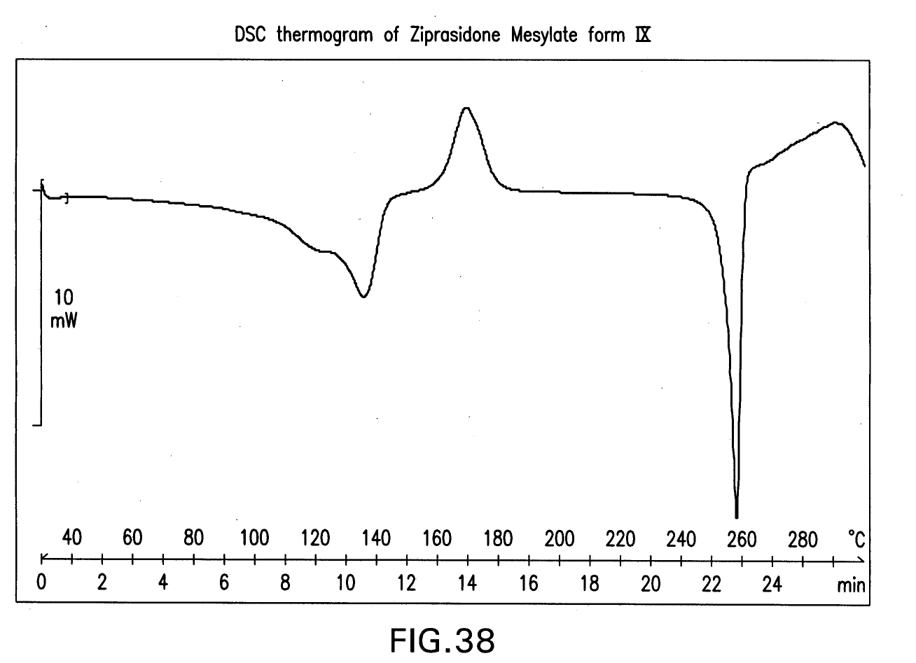

Form IX may also be characterized by a broad endotherm at the range of about 90° C. to about 143° C. of desolvation or dehydration as measured by differential scanning calorimetry (DSC), which is illustrated in FIG. 38.

Form IX may also be characterized by an exotherm at about 170° C., as measured by DSC, indicating recrystallization.

Form IX may also be characterized by a melting endotherm at about 257° C., as measured by DSC, indicating melting of the anhydrous form.

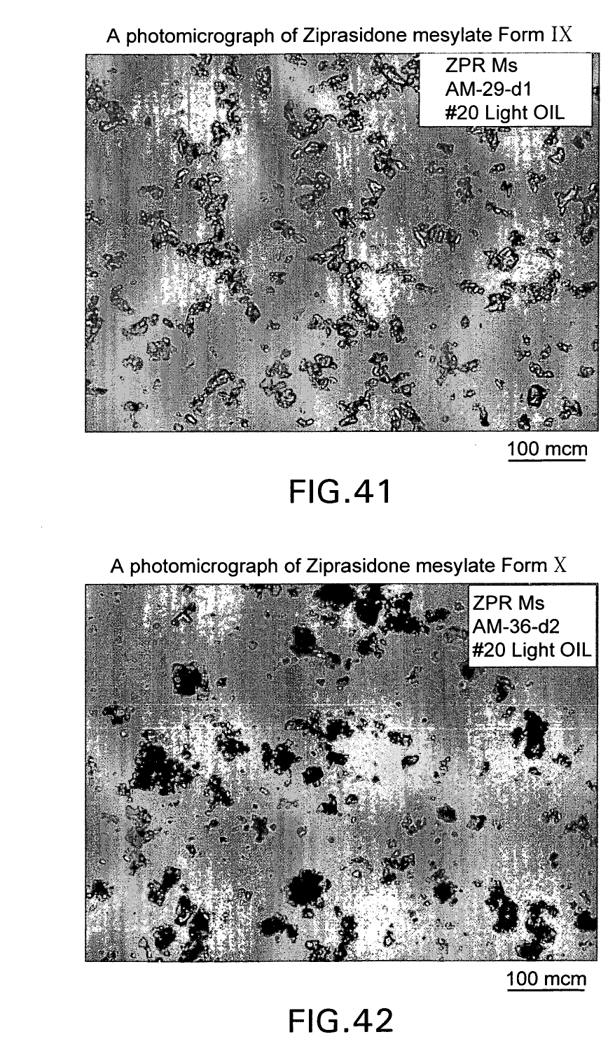

The morphology of Form IX particles is demonstrated in FIG. 41.

The present invention provides non-hygroscopic crystalline form of ziprasidone mesylate Form IX. Form IX was tested for water absorption after exposure for 10 days to 100% relative humidity at room temperature, as summarized in the following table:

TABLE 2
Water Absorption of Form IX.
Water content

| % RH | water Content (by Karl Fisher) |
|---|---|
| 100% at RT | 5.0 |
| Initial | 4.7 |

Another embodiment of the invention encompasses a process for preparing Form IX comprising slurring ziprasidone base, ethanol, water and methanesulfonic acid to obtain Form Ix.

Preferably, the ethanol used is in a ratio of 90% by volume to water.

Preferably, the reaction occurs while stirring at about room temperature for about 1 hour.

Another embodiment of the invention encompasses a process for preparing Form IX comprising: providing a slurry of ziprasidone base, ethanol and water, heating the slurry to a temperature of from about 40⁰ C to about 60⁰ C; combining the slurry with methanesulfonic acid; heating the mixture to a temperature of from about 60⁰ C to about 80⁰ C and cooling the mixture to about room temperature to obtain Ziprasidone mesylate Form IX.

Preferably, the ethanol used is in a ratio of about 85% to about 95% by volume to water, more preferably, the ethanol used is in a ratio of about 90% by volume to water.

Preferably, the slurry is heated to a temperature of about 50° C.

Preferably, the mixture is heated to a temperature of about 65° C.

Preferably, the mixture is maintained, while stirring, for about 1 hour.

Another embodiment of the invention encompasses a process for preparing Form IX comprising slurring zisperidone mesylate Form II and ethanol.

Preferably, the reaction occurs while stirring at about 50° C. for about 2 hours.

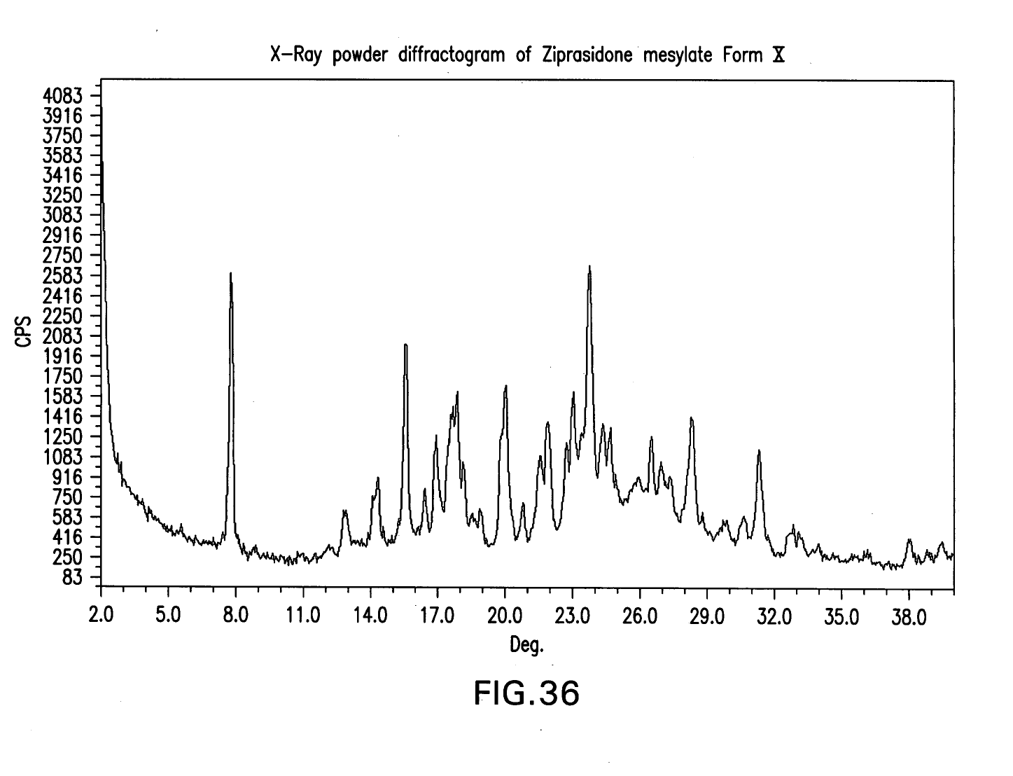

Another embodiment of the invention encompasses ziprasidone mesylate form, characterized by X-ray powder diffraction peaks at about 7.8, 15.6, 17.9, 20.0 and 23.8 degrees two-theta, ±0.2 degrees two-theta. This form is denominated Form X. Form X may be characterized further by X-ray powder diffraction peaks at about 16.9, 21.9, 23.0, 28.3 and 31.3 degrees two-theta, ±0.2 degrees two-theta. Form X may be substantially identified by FIG. 36.

Form X may be a THF solvate. This solvate may also be a hydrate, preferably, having about 0.9% water by weight, as measured by Karl Fisher.

Form X has a weight loss at the range of 25-180° C. as measured by TGA of about 3.4% by weight.

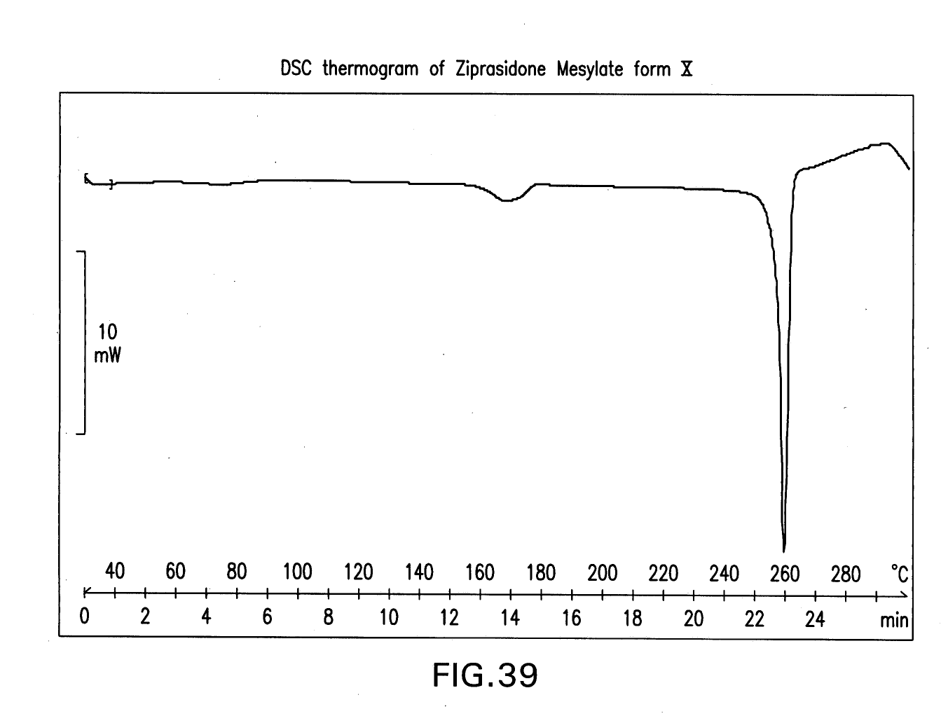

Form X may also be characterized by an endotherm at about 150-180° C., as measured by DSC, indicating desolvation or dehydration, which is illustrated in FIG. 39.

Form X may also be characterized by a melting endotherm at about 258° C., as measured by DSC, indicating melting of the anhydrous form.

The morphology of Form X particles is demonstrated in FIG. 42.

Another embodiment of the invention encompasses a process for crystallizing Form X from a mixture of methanesulfonic acid and a slurry of ziprasidone base, THF and water, at a temperature of about 5⁰ C.

Preferably, the THF used is in a ratio of 99% by volume to water.

Preferably, the reaction occurs while stirring for about 2 hours.

Ziprasidone mesylate Form X may then be recovered by any method known in art, such as filtration and drying the precipitate, preferably at about 40⁰ C-50⁰ C at a pressure below about 100 mmHg in a vacuum oven.

Another embodiment of the invention encompasses a process for preparing Form X by drying ziprasidone mesylate Form VII.

Preferably, wet ziprasidone mesylate Form VII is heated to a temperature of from about room temperature to about 40⁰ C, for a time sufficient to obtain ziprasidone mesylate Form X. Ziprasidone mesylate Form VII may be prepared as described above, preferably from a formic acid:methanol:water mixture. As one skilled in the art will appreciate, the time required to obtain ziprasidone mesylate Form X will vary depending upon, among other factors, the amount of wet ziprasidone mesylate Form VII to be dried and the drying temperature, and can be determined by taking periodic XRD's.

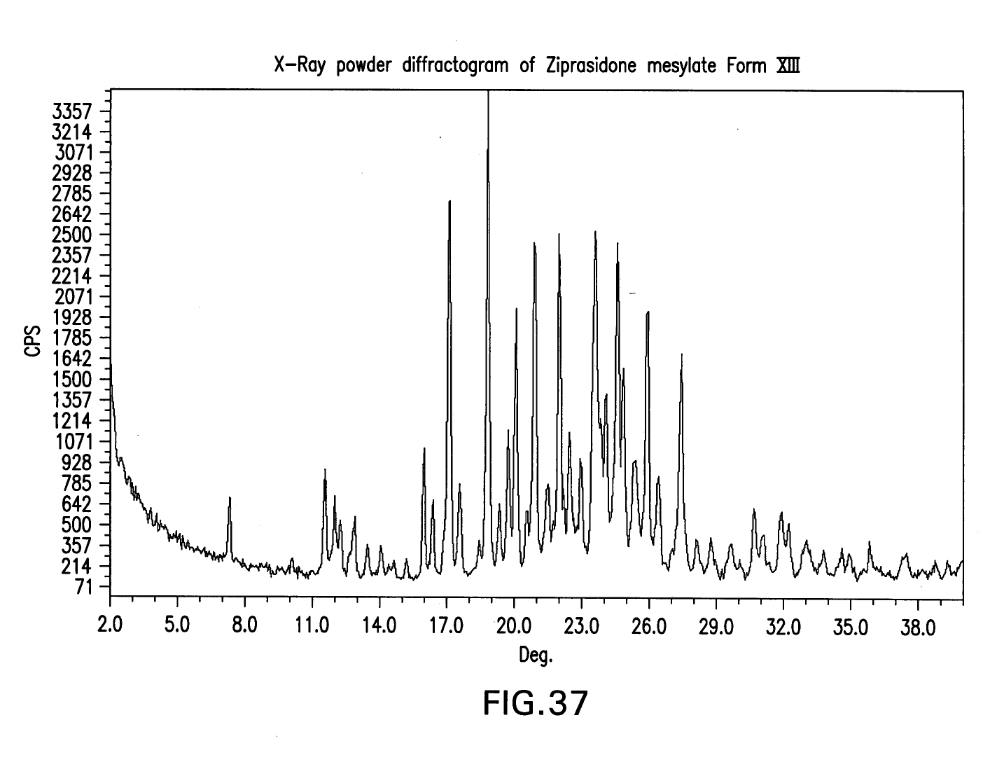

Another embodiment of the invention encompasses ziprasidone mesylate form, characterized by X-ray powder diffraction peaks at about 17.1, 18.9, 20.9, 22.0, 23.6 and 24.6 degrees two-theta, ±0.2 degrees two-theta. This form is denominated Form XIII. Form XIII may be characterized further by X-ray powder diffraction peaks at about 20.1, 24.9, 25.9 and 27.5 degrees two-theta, ±0.2 degrees two-theta. Form XIII may be substantially identified by FIG. 37.

Form XIII may be a monoethanolate. This solvate may also be a hydrate, preferably, having about 0.3% water by weight, as measured by Karl Fisher.

Form XIII has a weight loss at the range of 25-150° C. as measured by TGA of about 7.0% by weight.

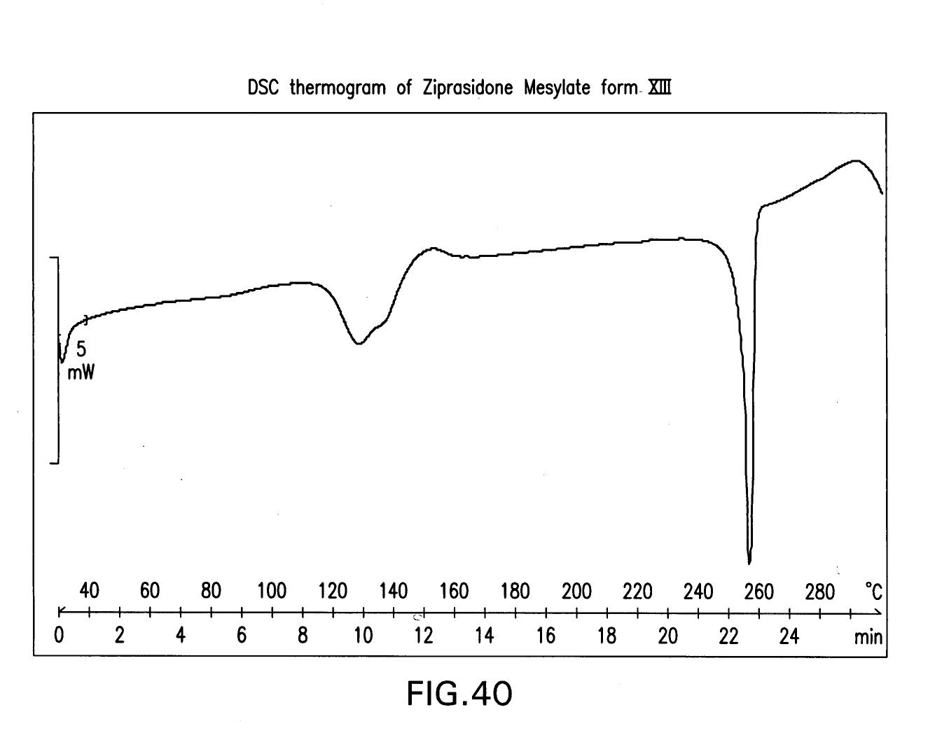

Form XIII may also be characterized by an endotherm at a range of about 110° C. to about 140° C., as measured by DSC, indicating desolvation, which is illustrated in FIG. 40.

Form XIII may also be characterized by an exotherm at about 150° C., as measured by DSC, indicating recrystallization.

Form XIII may also be characterized by a melting endotherm at about 256° C., as measured by DSC, indicating melting of the anhydrous form.

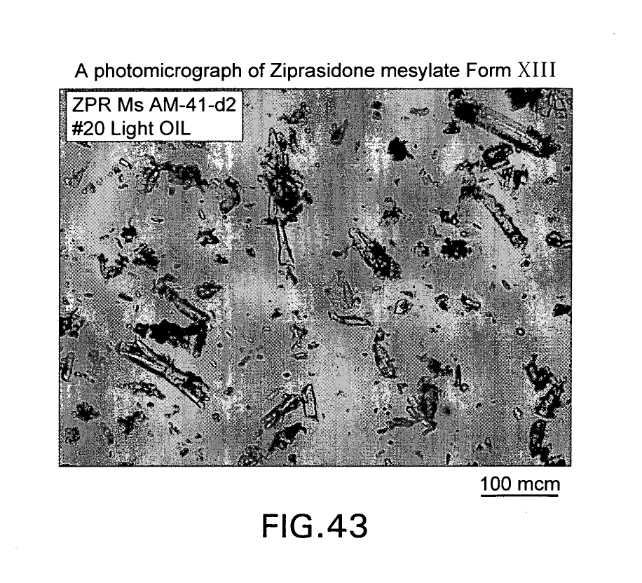

The morphology of Form XIII particles is demonstrated in FIG. 43.

Another embodiment of the invention encompasses a process for preparing Form XIII comprising: providing a slurry of ziprasidone base and absolute ethanol, heating the slurry to a temperature of from about 40⁰ C to about 60⁰ C; combining the slurry with methanesulfonic acid; heating the mixture to a temperature of from about 60⁰ C to about 80⁰ C and cooling the mixture to about room temperature to obtain Ziprasidone mesylate Form XIII.

Preferably, the slurry is heated to a temperature of about 50° C.

Preferably, the mixture is heated to a temperature of about 65° C.

Preferably, the mixture is maintained, while stirring, for about 30 minutes.

Preferably, after cooling to room temperature, the mixture is maintained, while stirring, for about 1 hour.

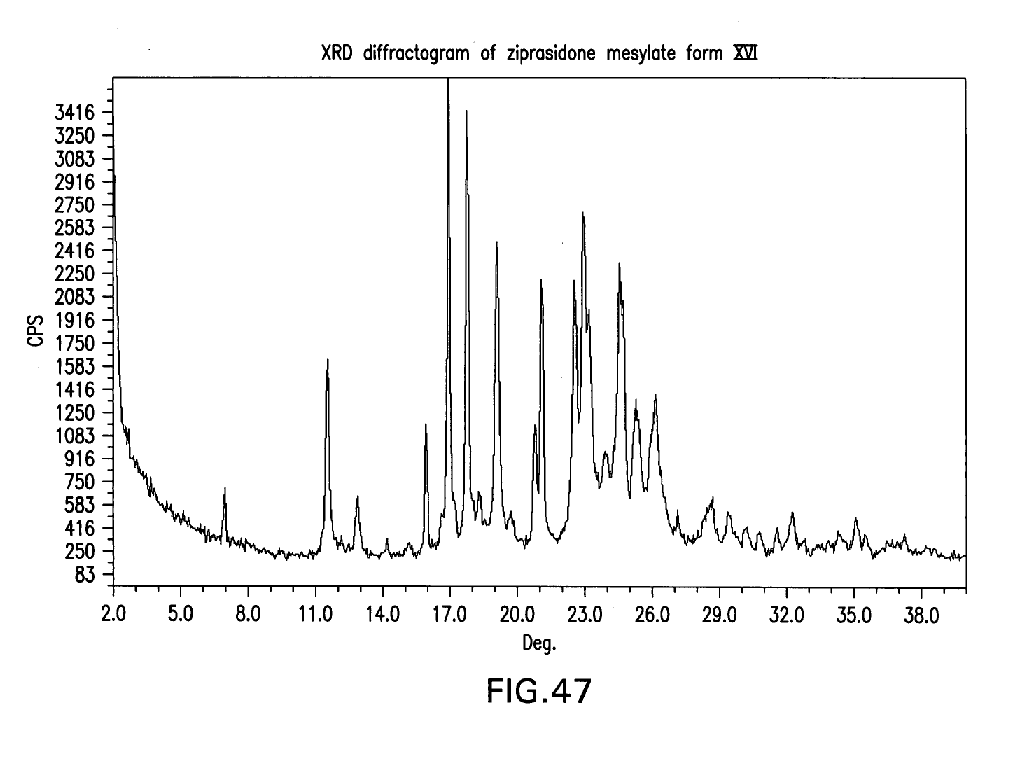

Another embodiment of the invention encompasses ziprasidone mesylate form, characterized by X-ray powder diffraction peaks at about 16.9, 17.7, 19.1, 21.1, 23.0 and 24.5 degrees two-theta, ±0.2 degrees two-theta. This form is denominated Form XVI. Form XVI may be characterized further by X-ray powder diffraction peaks at about 11.6, 15.9, 22.5 and 23.2 degrees two-theta, ±0.2 degrees two-theta. Form XVI may be substantially identified by FIG. 47.

Form XVI may be an ethylene glycol solvate. This solvate may also be a hydrate, preferably, having about 0.7% water by weight, as measured by Karl Fisher.

Form XVI has a weight loss at the range of 25-200° C. as measured by TGA of about 12.4% by weight.

Another embodiment of the invention encompasses a process for preparing Form XVI comprising: providing a slurry of ziprasidone base in ethylene glycol, heating the slurry to a temperature of from about 40⁰ C to about 60⁰ C; combining the slurry with methanesulfonic acid; cooling the mixture to a temperature of from about 60⁰ C to about room temperature, to obtain Ziprasidone mesylate Form XVI.

Preferably, the slurry is heated to a temperature of about 50° C.

Preferably, the mixture is cooled to a temperature of about 20° C.

Preferably, the mixture is maintained, while stirring, for about 30 minutes.

Preferably, after cooling, the mixture is maintained, while stirring, for about 1 hour.

Ziprasidone mesylate Form XVI may then be recovered by any method known in art, such as filtration and drying the precipitate, preferably at about 50⁰ C-70⁰ C at a pressure below about 100 mmHg in a vacuum oven.

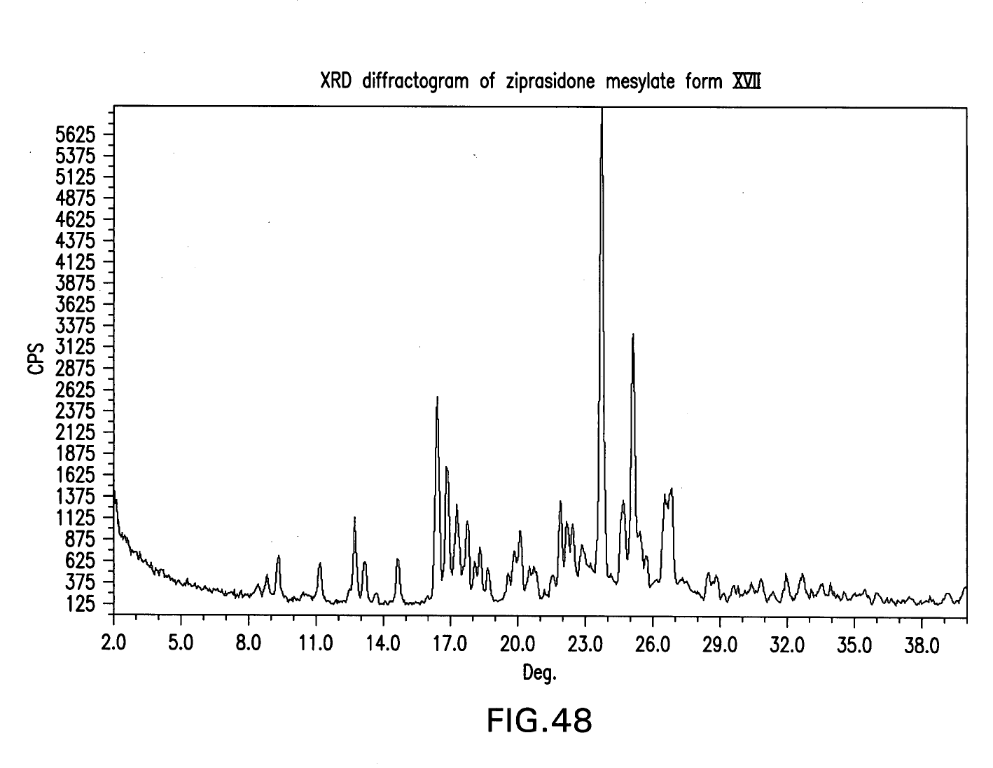

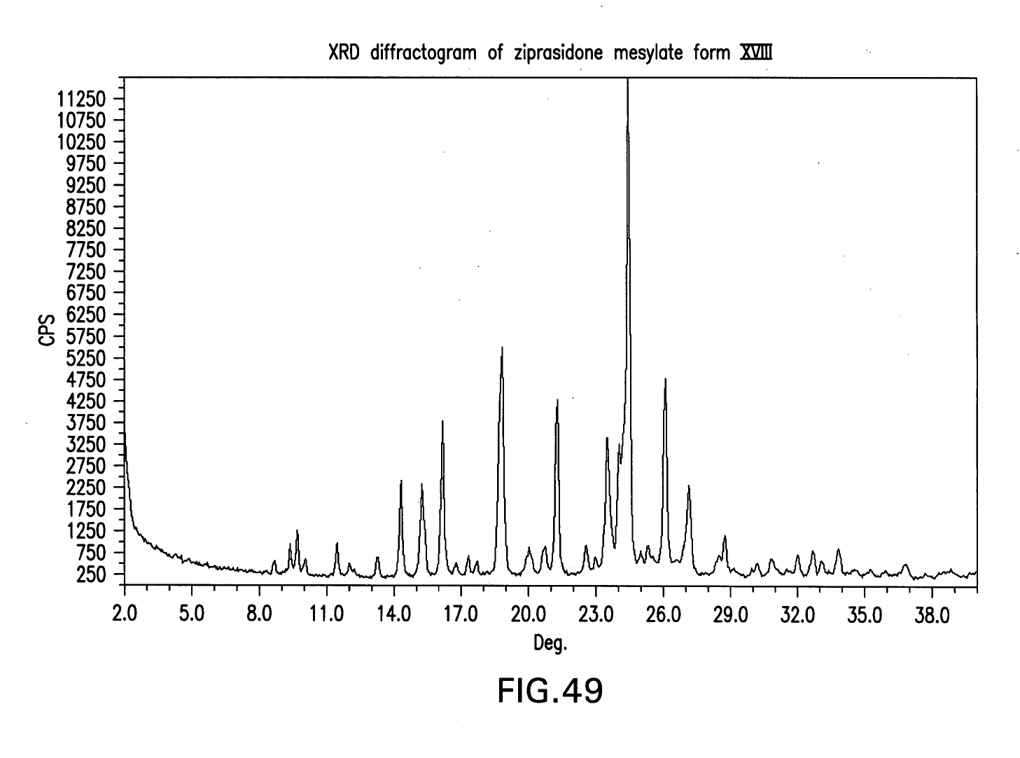

Another embodiment of the invention encompasses ziprasidone mesylate form, characterized by X-ray powder diffraction peaks at about 16.4, 16.9, 23.7, 25.1 and 26.9 degrees two-theta, ±0.2 degrees two-theta. This form is denominated Form XVII. Form XVII may be characterized further by X-ray powder diffraction peaks at about 12.7, 17.3, 20.1, 21.9 and 24.7 degrees two-theta, ±0.2 degrees two-theta. Form XVII may be substantially identified by FIG. 48.

Form XVII may be a 1,4-dioxane solvate. This solvate may also be a hydrate, preferably, monohydrate, having about 3% water by weight, as measured by Karl Fisher.

Form XVII has a weight loss at the range of 25-186° C. as measured by TGA of about 5.6% by weight.

Another embodiment of the invention encompasses a process for preparing Form XVII comprising slurring ziprasidone base, 1,4-dioxane and methanesulfonic acid to obtain Form XVII.

Preferably, the reaction occurs while stirring at about 25⁰ C for about 1 hour.

Ziprasidone mesylate Form XVII may then be recovered by any method known in art, such as filtration and drying the precipitate, preferably at about 50⁰ C-70⁰ C at a pressure below about 100 mmHg in a vacuum oven.

Another embodiment of the invention encompasses ziprasidone mesylate form, characterized by X-ray powder diffraction peaks at about 16.2, 18.8, 21.3, 24.4 and 26.1 degrees two-theta, ±0.2 degrees two-theta. This form is denominated Form XVIII. Form XVIII may be characterized further by X-ray powder diffraction peaks at about 14.3, 15.2, 23.5 and 24.0 degrees two-theta, ±0.2 degrees two-theta. Form XVIII may be substantially identified by FIG. 49.

Form XVIII may be produced as a hemihydrate, preferably having about 2% to about 3% water by weight, as measured by Karl Fisher.

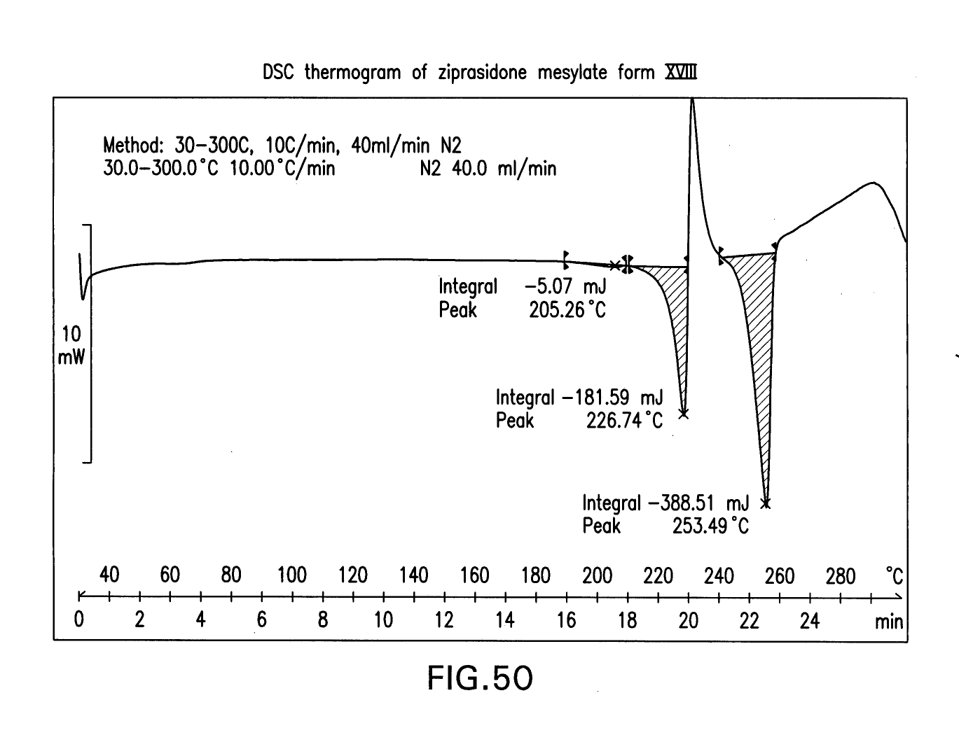

Form XVIII may also be characterized by an endotherm at about 205° C., as measured by DSC, which is illustrated in FIG. 50.

Form XVIII may also be characterized by an endotherm at about 227° C., as measured by DSC.

Form XVIII may also be characterized by an endotherm at about 253° C., as measured by DSC, which indicates the melting of the anhydrous form.

Form XVIII may also be characterized by a melting point in the range of from about 244° C. to about 255° C.

Exposing form XVIII to 80% humidity for 7 days at 30° C. showed transformation to trihydrate and about 8.2% of water content determined by Karl Fisher (see the following table):

TABLE 3
Water Absorption of Form XVIII.
INITIAL FORM: Form XVIII
DAYS: 7 days at 30° C.

| % RH | Water content (by KF) | Crystal form (by XRD) |
|---|---|---|
| 80 | 8.2 | Trihydrate |
| 100 | 9.1 | Trihydrate |
| Initial | 2.0 | XVIII |

Another embodiment of the invention encompasses a process for preparing Form XVIII by slurring ziprasidone mesylate anhydrous in water.

Preferably, the reaction occurs while stirring at about room temperature for about 15 minutes to about one week.

Ziprasidone mesylate Form XVIII may then be recovered by any method known in art, such as filtration or filtration under nitrogen and drying the precipitate, preferably at about 60⁰ C-70⁰ C at a pressure below about 100 mmHg in a vacuum oven.

Form XVIII may also be obtained by drying ziprasidone mesylate trihydrate under similar conditions.

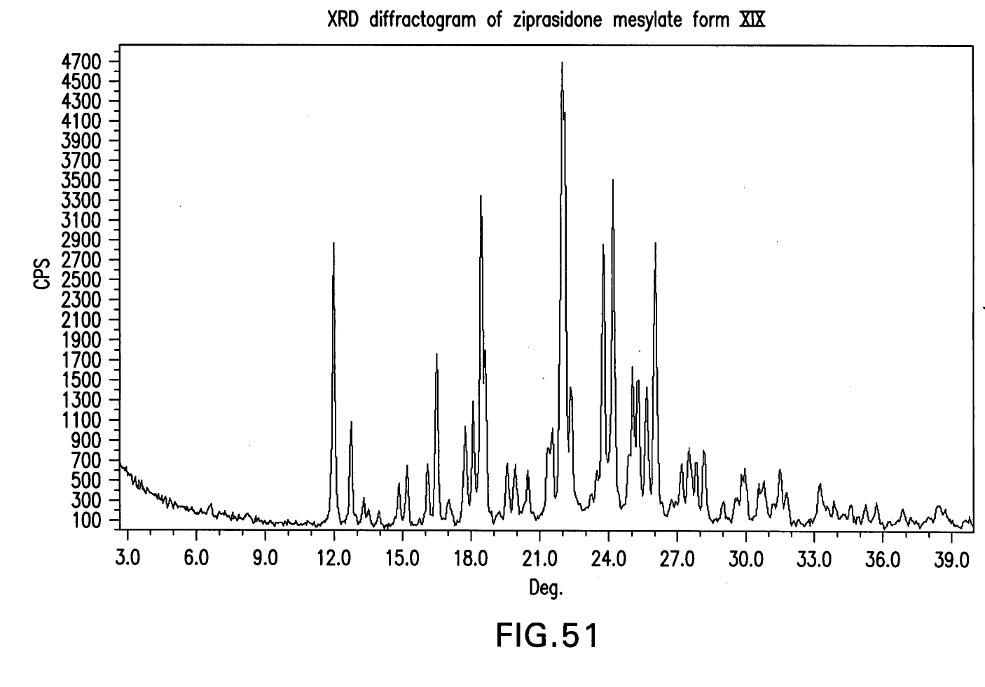

Another embodiment of the invention encompasses ziprasidone mesylate form, characterized by X-ray powder diffraction peaks at about 18.5, 22.0, 23.8, 24.2 and 26.1 degrees two-theta, ±0.2 degrees two-theta. This form is denominated Form XIX. Form XIX may be characterized further by X-ray powder diffraction peaks at about 12.0, 12.8, 16.5, 17.8 and 25.7 degrees two-theta, ±0.2 degrees two-theta. Form XIX may be substantially identified by FIG. 51.

Form XIX may be an acetic acid solvate. This solvate may also be a hydrate, preferably, having about 0.85% water by weight, as measured by Karl Fisher. The acetic acid content of Form XIX was determined by HPLC to be 10-12.5%.

Form XIX has a weight loss at the range of 40-160° C. as measured by TGA of about 12-13% by weight.

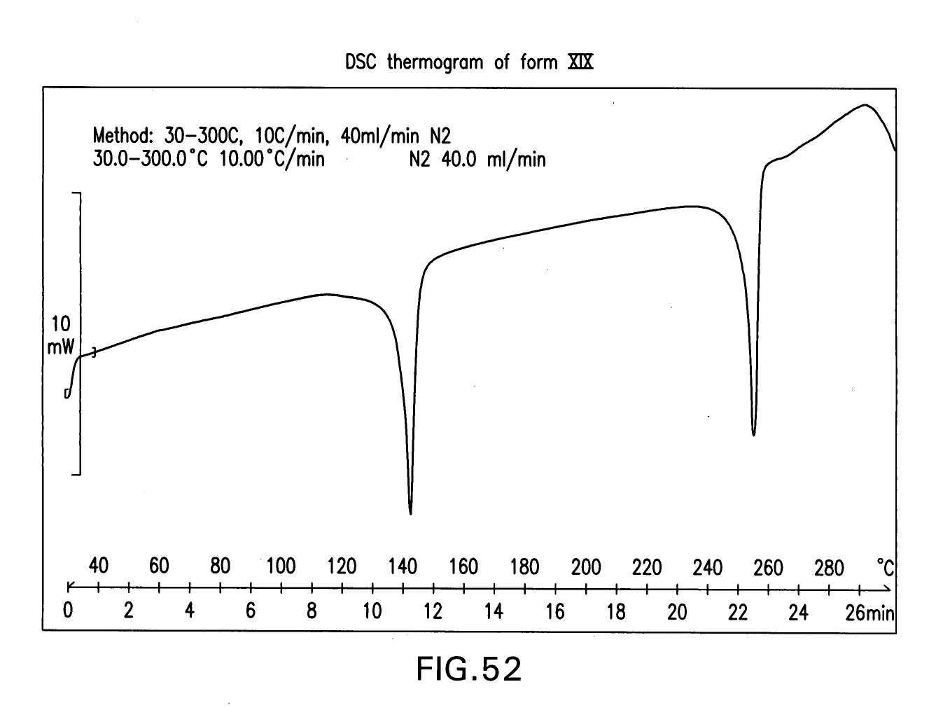

Comparison of the DSC thermogram (FIG. 52) to the TGA thermogram showed an endothermic peak (at about 140° C.) in the same range of the weight loss measured by TGA.

Another embodiment of the invention encompasses a process for crystallizing Form XIX from a mixture of methanesulfonic acid and a solution of ziprasidone base in acetic acid and an anti solvent such as isobutyl acetate.

Preferably, the reaction occurs while stirring at a temperature of from about 20⁰ C to about 40⁰ C for about 3 hours.

Alternatively, methanesulfonic acid is added to the solution only after cooling to room temperature.

Preferably, the acetic acid used is in a ratio of about 1:1.4 to about 2:1 by volume of isobutyl acetate used, more preferably about 1:1.4 acetic acid/isobutyl acetate.

Preferably, the acetic acid used is in a ratio of about 2:1 to about 6:1 by volume of Ziprasidone base used.

Ziprasidone mesylate Form XIX may then be recovered by any method known in art, such as filtration and drying the precipitate, preferably at about 60⁰ C-70⁰ C at a pressure below about 100 mmHg in a vacuum oven.

Another embodiment of the invention encompasses a process for preparing dihydrate needle crystals by slurrying of ziprasidone base, THF, water and methanesulfonic acid, at about room temperature.

Preferably, the THF used is in a ratio of 99% to about 80%, more preferably about 97% by volume to water.

Preferably, the reaction occurs while stirring for about 1 hour.

Another embodiment of the invention encompasses a process for preparing ziprasidone mesylate dihydrate lath crystals, comprising drying ziprasidone mesylate Form III, Form V or Form IX.

Preferably, wet ziprasidone mesylate Form III or Form V is heated to a temperature of about 80° C., for about 20 hours to obtain ziprasidone mesylate dihydrate lath crystals. Ziprasidone mesylate Form III or Form V may be prepared as described above. As one skilled in the art will appreciate, the time required to obtain ziprasidone mesylate Form X will vary depending upon, among other factors, the amount of wet ziprasidone mesylate Form III or Form V to be dried and the drying temperature, and can be determined by taking periodic XRD's.

Another embodiment of the invention encompasses a process for preparing ziprasidone mesylate anhydrous lath crystals, comprising drying ziprasidone mesylate Form IX.

Preferably, wet ziprasidone mesylate Form IX is heated to a temperature of from about 60° C. to about 100° C., more preferably to about 80° C., for more than about 20 hours. Ziprasidone mesylate Form IX may be prepared as described above.

Another embodiment of the invention encompasses a process for preparing a mixture of ziprasidone mesylate Form XIII and anhydrous ziprasidone mesylate lath crystals by drying ziprasidone Form II.

Preferably, wet ziprasidone mesylate Form II is heated to a temperature of from about 60° C. to about 100° C., more preferably to about 80° C., for more than about 20 hours. Ziprasidone mesylate Form II may be prepared as described above.

The present invention also encompasses pharmaceutical formulations comprising at least one of the crystalline forms of the present application and at least one pharmaceutically acceptable excipient. Another aspect of the present invention is a method for treating schizophrenia comprising administering a pharmaceutical composition comprising at least one of ziprasidone mesylate polymorphs disclosed in an therapeutically effective amount to treat, ameliorate, or reduce the symptoms associated with schizophrenia to a mammal (human) in need thereof.

In addition to the active ingredient(s), the pharmaceutical formulations of the present invention may contain one or more excipients. Excipients are added to the formulation for a variety of purposes.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flow ability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, ziprasidone and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste. Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

When preparing injectable (parenteral) pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules.

Excipients that are particularly well suited for direct compression tableting include microcrystalline cellulose; spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages can be conveniently presented in unit dosage form and prepared by any of the methods well-known in the pharmaceutical arts.

The dosage of GEODON may be used as guidance, and routine experimentation can be used to determine the appropriate dosage of the present invention. An oral dosage form of the present invention is preferably in the form of an oral capsule having a dosage of about 10 mg to about 160 mg, more preferably of about 20 mg to about 80 mg, and most preferably capsules of 20, 40, 60 and 80 mg. An injectable dosage preferably contains a dosage equivalent to about 5 to about 80 mg of ziprasidone base, and more preferably contains a dosage equivalent to about 10 to about 40 mg of ziprasidone base. Most preferably, an injectable dosage contains a dosage equivalent to about 20 to about 30 mg of ziprasidone base.

Having described the invention with reference to certain preferred embodiments, other embodiments will become apparent to one skilled in the art from consideration of the specification. The invention is further defined by reference to the following examples describing in detail the processes of the invention. It will be apparent to those skilled in the art that many modifications, both to materials and methods, may be practiced without departing from the scope of the invention.

### EXAMPLES

### Experimental

X-Ray powder diffraction data were obtained using a SCINTAG powder X-Ray diffractometer model X'TRA equipped with a solid state detector. Copper radiation of 1.5418 Å was used. A round aluminum sample holder with zero background was used. All peak positions are within ±0.2 degrees two theta.

DSC analysis was performed using a Mettler 821 Stare. The weight of the samples was about 3-6 mg; the samples were scanned at a rate of 10° C./min from 30° C. to at least 300° C. The oven was constantly purged with nitrogen gas at a flow rate of 40 ml/min. Standard 40 μl aluminum crucibles covered by lids with 3 holes were used.

TGA analysis was performed using a Mettler M3 thermogravimeter. The weight of the samples was about 8 mg; the samples were scanned at a rate of 10° C./min from 25° C. to 200° C. A blank was subtracted from the sample. The oven was constantly purged with nitrogen gas at a flow rate of 40 ml/min. Standard 150 μl alumina crucibles covered by lids with 1 hole were used.

Karl Fisher analysis was performed according to the known art.

Microscope: The material was dispersed in a light mineral oil before the measurement.

Preparation of Form I

### Example 1

To the solution of Ziprasidone base (5 g) in acetic acid (31.25 ml) and ethanol (18.75 ml) at 25° C., was added methanesulfonic acid (1 ml) and the mixture was stirred at that temperature for two hours. After two hours stirring the slurry was cooled to 20° C. to complete the precipitation; the obtained solid was filtered washed with ethanol and dried in a vacuum oven at 45° C. or in hood. The wet solid and the dried solid is ziprasidone mesylate Form I.

### Example 2

To the solution of Ziprasidone base (5 g) in mixture acetic acid/ethanol 1.6:1 (50 ml) at 25° C., was added methanesulfonic acid (1 ml) and the mixture was stirred at that temperature for two hours. After this the mixture was cooled to ˜20° C. and the solid was filtered washed with ethanol. The solid was dried in a vacuum oven at 45° C. or in hood. The wet solid and the dried solid is ziprasidone mesylate Form I.

### Example 3

To the solution of Ziprasidone base (30 g) in mixture acetic acid/MIBK 1:1 (375 ml) at ˜3° C., was added active carbon (3 g) and tonsil (3 g) and the slurry was stirred for 1 h. After this the mixture was filtrated at that temperature under nitrogen atmosphere and gently heated to about 20° C. Methanesulfonic acid (˜5 ml) was added drop-wise during about 30 min. and the mixture was stirred at that temperature for three hours. After this solid was filtered and washed with MIBK (40 ml). The solid was dried in a vacuum oven at 65° C. The wet solid is ziprasidone mesylate Form I.

### Example 4

To the solution of Ziprasidone base (30 g) in mixture acetic acid/iso-Butyl-acetate 1:1 (170 ml) at ˜3° C., was added active carbon (3 g) and tonsil (3 g) and the slurry was stirred for 30 min. After this the mixture was filtrated at that temperature under nitrogen atmosphere and gently heated to about 20° C. Methanesulfonic acid (˜5 ml) was added drop-wise during about 35 min. and the mixture was stirred at that temperature for two hours. After this the solid was filtered and washed with iso-Butyl-acetate (40 ml). The solid was dried in a vacuum oven at 65° C. The dried solid is ziprasidone mesylate Form I.

### Example 5

To the solution of Ziprasidone base (24 g) in acetic acid (150 ml) at ˜20° C., was added active carbon (2.4 g) and tonsil (2.4 g) and the slurry was stirred for 20 min. After this the mixture was filtrated at that temperature under nitrogen atmosphere. Methanesulfonic acid (˜4 ml) was added drop-wise during about 20 min. and the mixture was diluted by adding iso-propyl alcohol (140 ml) and stirred at that temperature for two hours. After this part of the obtained solid was filtered and washed with iso-propyl alcohol (20 ml). The solid was dried in a vacuum oven at 65° C. The dried solid is ziprasidone mesylate Form I.

Preparation of Form II

### Example 6

The slurry of Ziprasidone base (5 g) in ethanol 95% (50 ml) was heated to 50° C.; to this slurry while the temperature was maintained at 50° C. was added methanesulfonic acid (1 ml) over 5 min. Then the mixture was heated to 65° C. and the stirring was maintained at 65° C. for 30 min. Then the whole was cooled to the room temperature and the solid was filtered, washed with ethanol and dried. Part of the wet solid was dried on table and the second part was dried in oven under vacuum and at 45° C. Both dried samples are ziprasidone mesylate Form II (water content 1.7%).

### Example 7

To the slurry of Ziprasidone base (5 g) in ethanol 95% (50 ml) at 25° C. was added methanesulfonic acid (1 ml). Then the mixture was stirred with magnetic stirrer for two. Then the solid was filtered, washed with ethanol and dried. Part of the wet solid was dried on table and the second part was dried in oven under vacuum and at 45° C. The wet solid and both dried samples are ziprasidone mesylate Form II.

### Example 8

To the solution of Ziprasidone base (2 g) in mixture acetic acid and ethanol in a ratio of 1.6:1 (20 ml) at ˜5° C. was added methane sulfonic acid (0.4 ml) and the mixture was stirred at that temperature for two hours. After this the solid was filtered, washed with ethanol. The solid was dried in vacuum oven at 45° C. or in hood. The wet and dried solids are ziprasidone mesylate Form II.

### Example 9

To the slurry of Ziprasidone base (3 g) in ethanol tech. (95%) (30 ml) at 5° C., was added methane sulfonic acid (0.6 ml). The slurry was stirred at ˜5° C. for two hours and then, the temperature was allowed to reach ambient temperature. The solid was filtered, washed with methanol and dried at 45° C. The dried solid is ziprasidone mesylate Form II.

### Example 10

To the slurry of Ziprasidone base (3 g) in absolute ethanol (30 ml) at 5° C., was added methane sulfonic acid (0.6 ml). The slurry was stirred at ˜5° C. for two hours and then, the temperature was allowed to reach ambient temperature. The solid was filtered, washed with methanol and dried at 45° C. The wet and dried solid is ziprasidone mesylate Form II.

Preparation of Form III

### Example 11

Ziprasidone base (5 g) was dissolved in a mixture of formic acid, water and methanol in a ratio of 1:1:2 (50 ml) and the solution as heated with stirring to 50° C. To the solution methanesulfonic acid was added (1 ml) and then the mixture was heated to 65° C.; the stirring was maintained at 65° C. for 30 min. Then the solution was cooled to the room temperature; while the temperature was around 50° C. precipitation was observed. The solid was filtered, washed with water and dried on table and part in vacuum-oven at 45° C. The wet material and the dried materials is ziprasidone mesylate Form III (water content by K.F. 3.53%, 4.26% and 5.05%).

### Example 12

Ziprasidone mesylate Form V, wet solid, was dried in a laboratory hood or in vacuum oven at 45° C. The dried solid is ziprasidone mesylate Form III.

### Example 13

To the solution of Ziprasidone base (5 g) in a mixture of acetic acid and ethanol in a ratio of 5:3 (50 ml) was added methanesulfonic acid while the temperature was about 50° C. Then the mixture was heated to 65° C. and that temperature was maintained for 30 min. Then the reaction mixture was cooled to 20° C. The obtained solid was filtered, washed with ethanol and dried in hood. The dried solid is ZPR mesylate Form III.

### Example 14

The slurry of Ziprasidone base (2 g) in a mixture of ethanol 95% and water (18 ml: 2 ml) was cooled to about 5° C. While the temperature was ˜5° C. methane sulfonic acid (0.4 ml) was added and the stirring was maintained at that temperature for two hours. The solid was filtrated, washed with ethanol and dried in vacuum oven at 45° C. or in hood. The wet and dried solids are ZPR mesylate Form III.

### Example 15

The slurry of Ziprasidone base (3 g) in methanol HPLC grade (Baker) (30 ml) was heated to 50° C. While the temperature was about 50° C. methane sulfonic acid was added (0.6 ml) and then the reaction mixture was heated to 65° C. and stirred for 30 min. Then the mixture was cooled to 20° C. and stirred for one additional hour. The obtained solid was filtered, washed with methanol and dried (in vacuum oven at 45° C. or in hood). The wet and dried solids are ziprasidone mesylate Form III.

### Example 16

To the slurry of Ziprasidone base (5 g) in a mixture of tech. ethanol and water (40 ml/5 ml) at 50° C., was added methane sulfonic acid (1 ml). The resulted slurry was stirred at ˜65° C. for one hour, followed by cooling the mixture to room temperature and then filtering the solid, washing with tech. ethanol and draying in vacuum oven at 60° C. The wet and dried solid is ziprasidone mesylate Form III.

### Example 17

Ziprasidone mesylate Form II (0.5 g) was stirred with methanol (10 ml) at 50° C. for 2 hours. Then, the slurry was cooled to room temperature; the solid was filtered, washed with methanol and dried at 45° C. The wet and dried solids are a mixture of ziprasidone mesylate Form II and III.

Preparation of Form IV

### Example 18

Ziprasidone mesylate wet Form V was dried in hood; the dried solid is ziprasidone mesylate Form IV (water content by K.F. 5.4%).

Preparation of Form V

### Example 19

To the slurry of Ziprasidone base (5 g) in methanol (50 ml) at 50° C. was added methanesulfonic acid (1 ml) and than the slurry was heated to 65° C.; the heating was maintained with stirring at this temperature for 30 min., than the mixture was cooled to room temperature and the stirring was maintained for 16 hours. The solid was filtered, washed with methanol; the wet solid is ziprasidone mesylate Form V.

### Example 20

To the slurry of Ziprasidone base (5 g) in methanol (50 ml) at 25° C. was added methanesulfonic acid (1 ml) and than the slurry was stirred at 25° C. for two hours. The mixture was cooled to 20° C. and than the solid was filtered, washed with methanol; the wet solid was dried in hood at room temperature and in vacuum-oven. All samples (wet and dry) are ziprasidone mesylate Form V.

### Example 21

To slurry of Ziprasidone base (3 g) in methanol (30 ml) at 5° C. was added methane sulfonic acid (0.6 ml). The slurry was stirred at ˜5° C. for two hours and then the temperature was allowed to reach ambient temperature. The solid was filtered, washed with methanol and dried at 45° C. The dried solid is ziprasidone mesylate Form V.

### Example 22

The slurry of Ziprasidone base (10 g) in mixture toluene/methanol 8:1 (90 ml) was heated to ˜45o C; to this slurry methansulfonic acid (2 ml) was added over 10 min. The stirring was continued for 1 hour at 45o C than was cooled to room temperature and the solid was filtrated. The wet solid is Ziprasidone Mesylate Form V (water content 1.3%).

When the wet Form V was dried in vacuum-oven at 45° C. mixture of Form V and anhydrous lath crystals was obtained.

Preparation of Form VI

### Example 23

Dihydrate lath was heated at 160° C. for 30 minutes at oven. Transformation from dihydrate lath to Form VI was found

Preparation of Form VII

### Example 24

To the solution of Ziprasidone base in formic acid (12.5 ml), methanol (25 ml) and water (12.5 ml) at 25° C. was added methanesulfonic acid (1 ml) and the stirring was continued for two hours; after about 15 min. a precipitate is formed. After two hours stirring the mixture was cooled to 20° C. and the solid was filtered and washed with methanol. The wet and the dried material (drying was done at 45° C. in vacuum-oven or in hood at room temperature) is ziprasidone mesylate Form VII.

### Example 25

To the solution of Ziprasidone base (2 g) in a mixture of methanol (10 ml), formic acid (5 ml) and water (5 ml) at 5° C., was added methane sulfonic acid (0.4 ml). The slurry was stirred at ˜5° C. for two hours and then, the temperature was allowed to reach the ambient temperature. The solid was filtered, washed with water and dried at 45° C. The wet and dried solid is ziprasidone mesylate Form VII.

Preparation of Form VIII

### Example 26

Ziprasidone mesylate Form IX was dried in vacuum oven at 80° C. for less than 20 hours. The obtained solid is ziprasidone mesylate Form VIII.

Preparation of Form IX

### Example 27

The slurry of Ziprasidone base (5 g) in ethanol 95% (47.5 ml) and water (2.5 ml) was heated to 50° C. When the temperature was about 50° C. methane sulfonic acid (1 ml) was added and the reaction mixture was heated to 65° C. and stirred at this temperature for one hour. Then the reaction mixture was cooled to room temperature and stirred for an additional hour; then the solid was filtered, washed with ethanol 95%. The wet solid was analyzed by XRD and it is ziprasidone mesylate Form IX. Part of the wet solid was dried in vacuum oven at 45° C. and other part was left in hood at the room temperature. The two dried samples are ziprasidone mesylate Form IX (water content by K.F. 3.8%).

Ziprasidone mesylate Form IX may be prepared in a similar manner from the mixture ethanol: water in the ratio 85:15.

### Example 28

To the slurry of Ziprasidone base (3 g) in a mixture of ethanol and water (27 ml/3 ml) at ˜25° C., was added methane sulfonic acid (0.6 ml). The slurry was stirred at ˜25° C. for one hour. Then, the solid was filtered, washed with tech. ethanol dried at 45° C. The wet and dried solid is ziprasidone mesylate Form IX.

### Example 29

Ziprasidone mesylate Form II (0.5 g) was stirred with ethanol (5 ml) at 50° C. for 2 hours. Then, the slurry was cooled to room temperature; the solid was filtered, washed with ethanol and dried at 45° C. The wet and dried solids are ziprasidone mesylate Form IX.

### Example 30

Ziprasidone mesylate Form IX was exposed to 100% humidity at room temperature for 10 days. Before the exposure the water content was measured to be about 4.8%, while after the exposure the water content was measure to be about 5.0% water. The difference between water content before and after the exposure to 100% humidity is 0.2%.

Preparation of Form X

### Example 31

To the slurry of Ziprasidone base (2 g) in a mixture of THF and water in a ratio of 99:1 (20 ml) at ˜5o C was added methane sulfonic acid; the mixture was stirred at that temperature for 2 hours. The formed solid was filtered, washed with THF and dried at 45° C. under vacuum. The wet and dried solid is ZPR mesylate Form X (water content of the dried solid is 0.9% by K.F.).

### Example 32

ZPR mesylate Form VII wet solid prepared from a mixture of formic acid: methanol and water and then was dried in hood. The dried solid is ZPR mesylate Form X.

Preparation of Form XIII

### Example 33

To the slurry of Ziprasidone base (3 g) in absolute ethanol (AR) (30 ml) at a temperature of about 50° C., wad added methane sulfonic acid (0.6 ml). Then the slurry was heated at 65° C. for 30 min. and stirred with a mechanical stirrer. The reaction mixture was then cooled to room temperature and stirred for one hour. The solid was filtered, washed with ethanol and dried (in oven vacuum at 45° C. and in hood). The wet and dried solids are ZPR mesylate Form XIII (water content by K.F. 0.3%).

Preparation of Form XVI

### Example 34

The slurry of Ziprasidone base (3 g) in ethylene glycol (30 ml) was heated to 50° C. Then, methane sulfonic acid (0.6 ml) was added and the slurry was stirred for 30 minutes at 50° C., followed by cooling the reaction mixture to about 20° C. and stirring at that temperature for 1 hour. The solid was then filtered, washed with solvent and dried in vacuum-oven at 60° C. The wet and dried solid is ziprasidone mesylate Form XVI.

Preparation of Form XVII

### Example 35

To the slurry of Ziprasidone base (3 g) in 1,4-dioxane (30 ml) was added methane sulfonic acid (0.6 ml) at a temperature of 25° C. The stirring was applied for one hour at 23-25° C., followed by filtering the solid, washing with 1,4-dioxane (15 ml) and drying in vacuum-oven at 60° C. The wet and dried solid is ziprasidone mesylate Form XVII.

Preparation of Ziprasidone Mesylate Dihydrate Needle Crystals

### Example 36

To the slurry of Ziprasidone base (3 g) in THF (29.1 ml) and water (0.9 ml) at 25° C. was added methane sulfonic acid (0.6 ml). The slurry was stirred at that temperature for one hour, then was cooled to 20° C. and the solid was filtered, washed with THF water and dried in vacuum oven at 45° C. or in hood. The dried solids are ziprasidone mesylate dihydrate needles crystals.

Preparation of Ziprasidone Mesylate Dihydrate Lath Crystals

### Example 37

Ziprasidone mesylate Form III was dried in vacuum oven at 80° C. for 20 hours. The obtained solid is ziprasidone mesylate dihydrate lath crystals.

### Example 38

Ziprasidone mesylate Form V was dried in vacuum oven at 80° C. for 20 hours. The obtained solid is ziprasidone mesylate dihydrate lath crystals.

### Example 39

Ziprasidone mesylate Form IX was dried in vacuum oven at 80° C. for 20 hours. The obtained solid is ziprasidone mesylate dihydrate lath crystals.

Preparation of Form XVIII

### Example 40

The slurry of ziprasidone mesylate anhydrous (4 gr.) in water (40 ml) was stirred for one week at room temperature. After this time the solid was filtered, washed with water and dried in oven at 65° C. under vacuum. The dried solid was ziprasidone mesylate Form XVIII.

### Example 41

The slurry of ziprasidone mesylate anhydrous (2 gr) was stirred in water (15 ml) at room temperature for 15 minutes. After this the solid was filtered under nitrogen, washed with water, and dried at oven at 65° C. under vacuum. The dried solid was ziprasidone mesylate Form XVIII.

### Example 42

Ziprasidone mesylate trihydrate wet prepared by slurrying ziprasidone mesylate anhydrous in water, was dried in oven at 65° C., under vacuum. The dried solid was ziprasidone mesylate Form XVIII.

TABLE 4
Transformations by Heat.

| | HEAT TRANSFORMATION | | | |
|---|---|---|---|---|
| FORM | TEMP. | TIME (min) | RESULT (XRD) | K.F. |
| Dihydrate | 90 | 30 | Dihydrate Lath | 3.1 |
| Lath | | | | (t = 0 6.5%) |
| Dihydrate | 120 | 30 | Dihydrate Lath | 0.4% |
| Lath | | | | (t = 0 6.5%) |
| Dihydrate | 140 | 30 | Dihydrate Lath | 0.3% |
| Lath | | | | (t = 0 6.5%) |
| Dihydrate | 160 | 30 | Form VI | 0.2% |
| Lath | | | | (t = 0 6.5%) |
| Dihydrate | 90 | 30 | Dihydrate needle | 3% |
| needle | | | | (t = 0) |
| Dihydrate | 120 | 30 | Dihydrate needle | 0.5% |
| needle | | | | |
| Form II | 90 | 30 | Form II | |

### Example 43

To the solution of Ziprasidone base (10 g) in acetic acid-i-BuOAc (19.5 ml/27.5 ml) was added methane sulfonic acid (2.33 g=1 mol-equivalent). The temperature during the addition was between 25 to 30° C. After this the mixture was stirred at about 23° C. for 2.5 h. The solid was filtrated, washed with i-BuOAc (10 ml) and dried in vacuum-oven at 65° C. The dried material is Ziprasidone mesylate Form XIX (water content by K.F. 0.85%; acetic acid content by HPLC 12.23%).

Preparation of Ziprasidone Mesylate Anhydrous Lath Crystals

### Example 44

Ziprasidone mesylate Form IX was dried in vacuum oven at 80° C. for more than 20 hours. The obtained solid is Ziprasidone mesylate anhydrous lath crystals.

Preparation of a Mixture of Ziprasidone Mesylate Form XIII and Anhydrous Ziprasidone Mesylate Lath Crystals

### Example 45

Ziprasidone mesylate Form II was dried in vacuum oven at 80° C. for 20 hours. The obtained solid is a mixture of Form XIII and anhydrous lath crystals.

What is claimed is:

1. A crystalline form of ziprasidone mesylate, characterized by an X-ray powder diffraction pattern having peaks at about 11.7, 17.3, 23.5, 24.2 and 25.2 degrees two-theta ±0.2 degrees two-theta.

2. The crystalline form of claim 1, characterized by X-ray powder diffraction pattern having peaks at about 11.7, 17.3, 23.5, 24.2 and 25.2 degrees two-theta ±0.2 degrees two-theta.

3. The crystalline form of claim 2, further characterized by X-ray powder diffraction pattern having peaks at about 18.5, 20.7, 21.8, 22.7 and 25.7 degrees two-theta ±0.2 degrees two-theta.

4. The crystalline form of claim 3 characterized by an XRD pattern substantially identified by FIG. 1.

5. The crystalline form of claim 1, characterized by DSC having a melting endotherm at about 134° C.

6. The ziprasidone mesylate solvate of claim 1, wherein the crystal is a solvate of acetic acid.

7. The ziprasidone mesylate crystalline form of claim 6, wherein the solvate is also a hydrate.

8. The ziprasidone mesylate crystalline form of claim 7, having about 2.3% water by weight.

9. The ziprasidone mesylate crystalline form of claim 1, having a weight loss in the range of about 25° C. to about 180° C., of about 9.5% by weight.

10. A process for preparing the crystalline form of claim 1 comprising combining methanesulfonic acid with a solution of ziprasidone base, acetic acid and an anti solvent selected from the group consisting of: ethanol, isopropyl alcohol, methyl-isobutyl ketone and iso-butylacetate, at a temperature of about 20⁰ C to about 40⁰ C.

11. The process of claim 10, wherein the acetic acid used is in a ratio of about 1:1 to about 3:1 by volume of anti solvent used.

12. The process of claim 11, wherein the acetic acid used is in a ratio of about 1.6: by volume of anti solvent used.

13. The process of claim 10, wherein the anti solvent is ethanol.

14. The process of claim 10, wherein the total amount of solvents used is in a ratio of about 6 to 12 by volume of ziprasidone mesylate base used.

15. A crystalline form of ziprasidone mesylate, characterized by an X-ray powder diffraction pattern having peaks at about 17.1, 18.8, 21.0 and 23.7 degrees two-theta ±0.2 degrees two-theta.

16. The crystalline form of claim 15, further characterized by X-ray powder diffraction pattern having peaks at about 11.6, 20.1, 22.1, 24.2 and 27.5 degrees two-theta ±0.2 degrees two-theta.

17. The crystalline form of claim 16, characterized by an XRD pattern substantially identified by FIG. 6.

18. The ziprasidone mesylate crystalline form of claim 15, wherein the crystal is a solvate of ethanol.

19. The ziprasidone mesylate crystalline form of claim 18, wherein the solvate is also a hydrate.

20. The ziprasidone mesylate crystalline form of claim 19, having about 1.7% water by weight.

21. The ziprasidone mesylate crystalline form of claim 15, having a weight loss in the range of about 25° C. to about 150° C., of about 7.9% by weight.

22. A process of preparing the form of claim 15 comprising:
a. providing a slurry of ziprasidone base and ethanol;
b. heating the slurry to a temperature of from about 40⁰ C to about 60⁰ C;
c. combining the slurry with methanesulfonic acid to obtain a reaction mixture;
d. heating the mixture to a temperature of from about 60⁰ C to about 80⁰ C; and
e. cooling the mixture to about room temperature to obtain the ziprasidone mesylate form of claim 15.

23. The process of claim 22, wherein the ethanol used is in a ratio of above about 95% by volume to water.

24. The process of claim 23, wherein absolute ethanol is used.

25. The process of claim 23, wherein the slurry is heated to a temperature of about 50° C.

26. The process of claim 23, wherein the mixture is heated to a temperature of about 65° C.

27. A process for preparing the crystalline form of claim 15 comprising combining methanesulfonic acid with a slurry of ziprasidone base and ethanol, maintaining the slurry and recovering the crystalline form.

28. The process of claim 27, wherein the ethanol used is in a ratio of about 95% to about 99.9% by volume to water.

29. A process for preparing crystalline form of claim 15 comprising combining a mixture of methanesulfonic acid with a solution of ziprasidone base, acetic acid and ethanol at a temperature of about 0⁰ C to about 15⁰ C.

30. The process of claim 29, wherein the acetic acid is in a ratio of about 1:1.5 to about 3:1 by volume of ethanol used.

31. The process of claim 30, wherein the acetic acid is in a ratio of about 1.6:1 by volume of ethanol.

32. The process of claim 29, wherein the reaction occurs at about 5⁰ C.

33. A crystalline form of ziprasidone mesylate, characterized by an X-ray powder diffraction pattern having peaks at about 17.2, 19.0, 21.0, 24.3 and 24.9 degrees two-theta, ±0.2 degrees two-theta.

34. The crystalline form of claim 33, further characterized by X-ray powder diffraction pattern having peaks at about 11.9, 20.3, 23.0 and 26.5 degrees two-theta ±0.2 degrees two-theta.

35. The crystalline form of claim 34, characterized by an XRD pattern substantially identified by FIG. 26.

36. The ziprasidone mesylate crystalline form of claim 33, wherein the crystal is a solvate of methanol.

37. The ziprasidone mesylate crystalline form of claim 36, wherein the solvate is also a hydrate.

38. The ziprasidone mesylate crystalline form of claim 37, having about 2.3% water by weight.

39. The ziprasidone mesylate crystalline form of claim 33, having a weight loss in the range of about 25° C. to about 180° C., of about 7.9% by weight.

40. A process for preparing the crystalline form of claim 33 comprising combining methanesulfonic acid with a solution of ziprasidone base, formic acid, and a solvent selected from the group consisting of C₁₋₅ alcohol and water, at a temperature of about 5⁰ C to about room temperature.

41. The process of claim 40, wherein the solvent is formic acid and mixtures thereof with methanol and water.

42. The process of claim 40, wherein the formic acid used is in a ratio of about 1:1 to about 1:3 by volume of methanol used.

43. The process of claim 42, wherein the formic acid used is in a ratio of about 1:2 by volume of methanol used.

44. The process of claim 43, wherein the solvents ratio used is 1:2:1 formic acid/methanol/water.

45. A crystalline form of ziprasidone mesylate, characterized by an X-ray powder diffraction pattern having peaks at about 17.1, 18.7, 20.9, 23.8 and 24.3 degrees two-theta ±0.2 degrees two-theta.

46. The crystalline form of claim 45, characterized by X-ray powder diffraction pattern having peaks at about 17.1, 18.7, 20.9, 23.8 and 24.3 degrees two-theta ±0.2 degrees two-theta.

47. The crystalline form of claim 46, further characterized by X-ray powder diffraction pattern having peaks at about 11.7, 20.0, 21.0 and 25.8 degrees two-theta ±0.2 degrees two-theta.

48. The crystalline form of claim 47, characterized by an XRD pattern substantially identified by FIG. 35.

49. The crystalline form of claim 45, characterized by DSC having a broad endotherm at the range of about 90° C. to about 143° C.; an exotherm at about 170° C.; and a melting endotherm at about 257° C.

50. The ziprasidone mesylate crystalline form of claim 45, wherein the crystal is a solvate of ethanol.

51. The ziprasidone mesylate crystalline form of claim 50, wherein the solvate is also a sesquihydrate.

52. The ziprasidone mesylate crystalline form of claim 51, having about 4.7% water by weight.

53. The ziprasidone mesylate crystalline form of claim 45, having a weight loss in the range of about 25° C. to about 150° C., of about 6.7% by weight.

54. A process for preparing the form of claim 45 comprising slurrying ziprasidone base, ethanol, water and methanesulfonic acid.

55. The process of claim 54, wherein the ethanol used is in a ratio of 90% by volume to water.

56. The process of claim 54, wherein the slurry is carried out at about room temperature.

57. A process of preparing the form of claim 45 comprising:
a. providing a slurry of ziprasidone base and ethanol;
b. heating the slurry to a temperature of from about 40⁰ C to about 60⁰ C;
c. combining the slurry with methanesulfonic acid;
d. heating the mixture to a temperature of from about 60⁰ C to about 80⁰ C; and
e. cooling the mixture to about room temperature to obtain the ziprasidone mesylate form of claim 45.

58. The process of claim 57, wherein the ethanol used is in a ratio of about 85% to about 95% by volume to water.

59. The process of claim 58, wherein the ethanol used is in a ratio of about 90% by volume to water.

60. The process of claim 57, wherein the slurry is heated to a temperature of about 50° C.

61. The process of claim 57, wherein the mixture is heated to a temperature of about 65° C.

62. A process for preparing the form of claim 45 comprising slurring the zisperidone mesylate form of claim 15 and ethanol.

63. The process of claim 62, wherein the reaction occurs at 50⁰ C.

64. A crystalline form of ziprasidone mesylate, characterized by an X-ray powder diffraction pattern having peaks at about 18.5, 22.0, 23.8, 24.2 and 26.1 degrees two-theta, ±0.2 degrees two-theta.

65. The crystalline form of claim 64, further characterized by X-ray powder diffraction pattern having peaks at about 12.0, 12.8, 16.5, 17.8 and 25.7 degrees two-theta ±0.2 degrees two-theta.

66. The crystalline form of claim 65, characterized by an XRD pattern substantially identified by FIG. 51.

67. The ziprasidone mesylate crystalline form of claim 64, wherein the crystal is a solvate of acetic acid.

68. The ziprasidone mesylate solvate of claim 67, having about 10-12.5% acetic acid content.

69. The ziprasidone mesylate crystalline form of claim 67, wherein the solvate is also a hydrate.

70. The ziprasidone mesylate crystalline form of claim 69, having about 0.85% water by weight.

71. The ziprasidone mesylate crystalline form of claim 64, having a weight loss in the range of about 40° C. to about 160° C., of about 12-13% by weight.

72. A process for crystallizing the form of claim 64 comprising combining methanesulfonic acid with a solution of ziprasidone base in acetic acid and an anti solvent.

73. The process of claim 72, wherein the reaction occurs at a temperature of from about 20⁰ C to about 40⁰ C.

74. The process of claim 72, wherein methanesulfonic acid is added to the solution only after cooling to room temperature.

75. The process of claim 72, wherein the acetic acid used is in a ratio of about 1:1.4 to about 2:1 by volume of isobutyl acetate used.

76. The process of claim 75, wherein the acetic acid used is in a ratio of about 1:1.4 by volume of isobutyl acetate used.

77. The process of claim 72, wherein the acetic acid used is in a ratio of about 2:1 to about 6:1 by volume of Ziprasidone base used.

78. A crystal form of ziprasidone mesylate characterized by an X-ray powder diffraction pattern having peaks at about:
20.9, 21.3, 24.0, 24.5 and 25.8 degrees two-theta, ±0.2 degrees two-theta;
17.1, 18.9, 22.7, 23.6 and 24.3 degrees two-theta, ±0.2 degrees two-theta;
22.1, 25.5, 26.8, 27.1 and 27.5 degrees two-theta, ±0.2 degrees two-theta;
15.1, 23.0, 23.5 and 23.8 degrees two-theta, ±0.2 degrees two-theta;
17.1, 18.7, 23.8 and 24.4 degrees two-theta ±0.2 degrees two-theta;
7.8, 15.6, 17.9, 20.0 and 23.8 degrees two-theta ±0.2 degrees two-theta;
17.1, 18.9, 20.9, 22.0, 23.6 and 24.6 degrees two-theta ±0.2 degrees two-theta;
16.9, 17.7, 19.1, 21.1, 23.0 and 24.5 degrees two-theta ±0.2 degrees two-theta;
16.4, 16.9, 23.7, 25.1 and 26.9 degrees two-theta, ±0.2 degrees two-theta; or
16.2, 18.8, 21.3, 24.4 and 26.1 degrees two-theta, ±0.2 degrees two-theta.

79. A process for preparing dihydrate needle crystals comprising combining methanesulfonic acid with a slurry of ziprasidone base and THF, maintaining the slurry at about room temperature and recovering the crystalline form.

80. The process of claim 79, wherein the THF used is in a ratio of 99% to about 80% by volume to water.

81. The process of claim 80, wherein the THF used is in a ratio of 97% by volume to water.

82. A process for preparing ziprasidone mesylate dihydrate lath crystals, comprising drying ziprasidone mesylate crystal forms characterized by an X-ray powder diffraction pattern having peaks at about:
20.9, 21.3, 24.0, 24.5 and 25.8 degrees two-theta, ±0.2 degrees two-theta;
22.1, 25.5, 26.8, 27.1 and 27.5 degrees two-theta, ±0.2 degrees two-theta; or
the crystal form of claim 45.

83. The process of claim 82, wherein the drying is at a temperature of about 80° C.

84. A process for preparing ziprasidone mesylate anhydrous lath crystals, comprising drying the ziprasidone mesylate form of claim 45.

85. The process of claim 84, wherein the ziprasidone mesylate form of claim 45 is dried at a temperature of from about 60° C. to about 100° C.

86. The process of claim 85, wherein the ziprasidone mesylate form of claim 45 is dried at a temperature of about 80° C.

87. The process of claim 84, wherein the ziprasidone mesylate form of claim 45 is dried for more than about 20 hours.

88. A process for preparing a mixture of ziprasidone mesylate crystal form characterized by an X-ray powder diffraction pattern having peaks at about 17.1, 18.9, 20.9, 22.0, 23.6 and 24.6 degrees two-theta ±0.2 degrees two-theta and anhydrous ziprasidone mesylate lath crystals, by drying the ziprasidone mesylate crystal form of claim 15.

89. The process of claim 88, wherein the ziprasidone mesylate form of claim 15 is dried at a temperature of from about 60° C. to about 100° C.

90. The process of claim 89, wherein the ziprasidone mesylate form of claim 15 is dried at a temperature of about 80° C.

91. The process of claim 88, wherein the ziprasidone mesylate form of claim 15 is dried for more than about 20 hours.

92. A pharmaceutical composition comprising the ziprasidone mesylate of any of claims 1, 15, 33, 45 or 64, and a pharmaceutically acceptable excipient.

93. A method of treating a schizophrenia comprising administering a therapeutically effective amount of the pharmaceutical composition of claim 92.
